SEQUENCE LISTING (40 sequences)

SEQ ID NO: 1 - DNA, 51 bp, Cannabis sativa
ctagtttgtttgttgagggattagcatatcaaattagataatgataataga

SEQ ID NO: 2 - DNA, 51 bp, Cannabis sativa
ttcaaacccacctccggaccctacctattcggacatccaaacatgccctta

SEQ ID NO: 3 - DNA, 51 bp, Cannabis sativa
atgttatggtgctcatttttaatctgtctctgttctatttcattttatgct

SEQ ID NO: 4 - DNA, 51 bp, Cannabis sativa
ttgcatttaataaataaaaatatacgtcttaaaatcttacccgtttttcta

SEQ ID NO: 5 - DNA, 51 bp, Cannabis sativa
tcggatgttcaatatttttgctgcccgaagacataactgaggtactttctt

SEQ ID NO: 6 - DNA, 51 bp, Cannabis sativa
ttattattattaaaatttttattgtgtacattaaatttcacaatttaaatt

SEQ ID NO: 7 - DNA, 51 bp, Cannabis sativa
agaaattgaaagaaagaaaaaaagaagaaaaaacaagggttgcaactgtaa

SEQ ID NO: 8 - DNA, 51 bp, Cannabis sativa
tgagcttagcaagctcaacatccttactcatcttcttcctatacatctcca

SEQ ID NO: 9 - DNA, 51 bp, Cannabis sativa
cgaaagatttgggaaggaatcaacaccaagtaattctcttttgtttagaaa

SEQ ID NO: 10 - DNA, 51 bp, Cannabis sativa
accgccggagaactgataagccggtcgaccggttagtagcagaagataaac

SEQ ID NO: 11 - DNA, 51 bp, Cannabis sativa
agatatatgaggaagaaaattaaagaagaggagggtcaagatagtgtcatt

SEQ ID NO: 12 - DNA, 51 bp, Cannabis sativa
agaattgttagaaacaacgttattgtaaatcttaatagccaataatagttc

SEQ ID NO: 13 - DNA, 51 bp, Cannabis sativa
atcattgaagaaatagtaagaaaaaaattgcatagaccagaagatatcgta

SEQ ID NO: 14 - DNA, 51 bp, Cannabis sativa
atagagtttaataacgctaatcctcttactgattgacatcaaatgttcgtt

SEQ ID NO: 15 - DNA, 51 bp, Cannabis sativa
agggctcaaaatttatgaagagaaaaagaggagaattcgaaatctatagaa

SEQ ID NO: 16 - DNA, 51 bp, Cannabis sativa
ttacaggcacattaaaatatgcagcaacaatctggttgtttgaaccgtgag

SEQ ID NO: 17 - DNA, 51 bp, Cannabis sativa
gctctcatatttctcaatatatgtttgaggccattgtaatccatataggct

SEQ ID NO: 18 - DNA, 51 bp, Cannabis sativa
tattgattcaatgatttttggcttttggatttccattcacggtggaatata

SEQ ID NO: 19 - DNA, 51 bp, Cannabis sativa
agctcaagtggtgagtttaggcggggttgtggtattagaccttttacgtat

SEQ ID NO: 20 - DNA, 51 bp, Cannabis sativa
tgtcctttccctcgcaaatacagtgctgagtggctctgttcacattagtaa

SEQ ID NO: 21 - DNA, 51 bp, Cannabis sativa
tctcccactacaaaatttgtgcacatattcactaacattgctaaaatgcat

SEQ ID NO: 22 - DNA, 51 bp, Cannabis sativa
agttactacttctataatttagccttaaataactatgctcatactaactat

SEQ ID NO: 23 - DNA, 51 bp, Cannabis sativa
cacttccacgtaatactctttattttccaattctacccctaacctttaata

SEQ ID NO: 24 - DNA, 51 bp, Cannabis sativa
ccacaacattccaaatgtaagattaactaacagaacatatcaaattttgtc

SEQ ID NO: 25 - DNA, 51 bp, Cannabis sativa
tgtatttgcttccttcgatgttcaaggccacttgtgagtgtacaagcgata

SEQ ID NO: 26 - DNA, 51 bp, Cannabis sativa
ggaaatgggattaatcatatgatttcttcggaatctaaaaacaatactaca

SEQ ID NO: 27 - DNA, 51 bp, Cannabis sativa
gtggatcatggtgtaatggtgtcactgtaccagccggaccgaaccaagcat

SEQ ID NO: 28 - DNA, 51 bp, Cannabis sativa
gcaaatcttgagcttcaatcacattcactctaacataccaaagctttggag

SEQ ID NO: 29 - DNA, 51 bp, Cannabis sativa
aaaaaccttcatttctagataatatcgagtcgtgtatatataccatgcaat

SEQ ID NO: 30 - DNA, 51 bp, Cannabis sativa
aactatttataattgcctccactatgaaataaaagtctaccaaagcatatc

SEQ ID NO: 31 - DNA, 51 bp, Cannabis sativa
gtaatggtcaggcctccacccaagtctgtgcatttgaaggtgcagtacaag

SEQ ID NO: 32 - DNA, 51 bp, Cannabis sativa
atagatttacatacctatatttatagaattttagtataaagtaatatgagt

SEQ ID NO: 33 - DNA, 51 bp, Cannabis sativa
tttatgtacaaatatgtgattatttcaattctcattttccattgtgctact

SEQ ID NO: 34 - DNA, 51 bp, Cannabis sativa
ttgaactaacagtacaaggaacaccgaaatctcagtcaaaagcacaaaccc

SEQ ID NO: 35 - DNA, 51 bp, Cannabis sativa
tttctgctcgggttatttgtgaagcaattttttacggttttcatatacttg

SEQ ID NO: 36 - DNA, 51 bp, Cannabis sativa
aaagaccgtatacagctgtaggaacgcttcgcgaccagttaatttatcctc

SEQ ID NO: 37 - DNA, 51 bp, Cannabis sativa
tctcacagagggaagcagaattgagggcattgcagctctatgggaaaggtg

SEQ ID NO: 38 - DNA, 51 bp, Cannabis sativa
catccatccaatcttttttccattccttttggtgttgggatagttgttata

SEQ ID NO: 39 - DNA, 51 bp, Cannabis sativa
agaagatctaagtactaaactaatgattcttctcatacgtgtacatgttac

SEQ ID NO: 40 - DNA, 51 bp, Cannabis sativa
ttccaggattgacacgaattttatcgaagcattcagcaactcttaatgcaa

(19) United States
(12) Patent Application Publication
(10) Pub. No.: US 2024/0117450 A1
(43) Pub. Date: Apr. 11, 2024

(54) POWDERY MILDEW MARKERS FOR CANNABIS

(71) Applicant: Phylos Bioscience, Inc., Portland, OR (US)

(72) Inventors: Erica Bakker, Portland, OR (US); Alisha Holloway, Portland, OR (US)

(73) Assignee: Phylos Bioscience, Inc., Portland, OR (US)

(21) Appl. No.: 18/276,615

(22) PCT Filed: Feb. 4, 2022
(86) PCT No.: PCT/US2022/015318

§ 371 Date: Aug. 9, 2023

Related U.S. Application Data
(60) Provisional application No. 63/147,709, filed on Feb. 9, 2021.

(51) Int. Cl. C12Q 1/6895; A01H 1/00; A01H 6/28

(52) U.S. Cl. CPC C12Q 1/6895; A01H 1/1255; A01H 6/28; C12Q 2600/13; C12Q 2600/166

(57) ABSTRACT

Provided herein is the identification of markers and genes associated with powdery mildew susceptibility and resistance in Cannabis. The markers are useful for breeding Cannabis plants having resistance to powdery mildew, and methods for selecting plants by obtaining nucleic acids and detecting one or more markers that indicate resistance to powdery mildew to establish plant lines having such resistance are provided.

### SEQUENCE LISTING REFERENCE

Pursuant to 37 CFR §§ 1.821-1.825, a Sequence Listing in the form of an ASCII-compliant text file (entitled “2006-WO1_ST25_Sequence_listing.txt” created on Jan. 28, 2022 and 6,197 bytes in size), which will serve as both the paper copy required by 37 CFR § 1.821(c) and the computer readable form (CRF) required by 37 CFR § 1.821(e), is submitted concurrently with the instant application. The entire contents of the Sequence Listing are incorporated herein by reference.

### BACKGROUND OF THE INVENTION

This application is directed to the field of powdery mildew in Cannabis. In particular, identifying genes and markers involved in susceptibility and resistance to powdery mildew.

Powdery mildew is a common fungal disease that affects most plants, including Cannabis. Outbreaks of powdery mildew have the potential to infect and destroy an entire greenhouse or outdoor grow operation, thus leading to significant loss of commercial opportunity. Thus, the ability to suppress powdery mildew by preventing its infection or stopping ongoing progression is of significant utility.

The invention described herein provides a solution through the identification of markers and genes that can be used to develop powdery mildew-resistant Cannabis varieties.

### SUMMARY OF THE INVENTION

The present teachings relate to methods of selecting plants having resistance to powdery mildew. In an embodiment, a method for selecting one or more cannabis plants comprising resistance to powdery mildew is provided. The method comprises, (i) obtaining nucleic acids from a sample cannabis plant or its germplasm; (ii) detecting one or more markers that indicate resistance to powdery mildew, and (iii) indicating resistance to powdery mildew. In an embodiment the method further comprises selecting the one or more plants indicating resistance to powdery mildew. In an embodiment, the one or more markers comprises a polymorphism relative to a reference genome at nucleotide position: (a) 15,287,266 on chromosome 1; (b) 15,368,894 on chromosome 1; (c) 95,466,762 on chromosome 2; (d) 119,379 on chromosome 2 (e) 149,520 on chromosome 2 (f) 181,346 on chromosome 2; (g) 325,930 on chromosome 2; (h) 396,437 on chromosome 2; (i) 494,430 on chromosome 2; (j) 887,683 on chromosome 2; (k) 1,114,228 on chromosome 2; (l) 1,262,671 on chromosome 2; (m) 1,368,060 on chromosome 2; (n) 1,430,121 on chromosome 2; (o) 1,434,872 on chromosome 2; (p) 1,489,951 on chromosome 2; (q) 1,791,640 on chromosome 2; (r) 2,136,725 on chromosome 2; (s) 3,436,566 on chromosome 4; (t) 3,502,761 on chromosome 4; (u) 3,911,993 on chromosome 4; (v) 3,946,006 on chromosome 4; (w) 3,995,523 on chromosome 4; (x) 4,065,757 on chromosome 4; (y) 6,578,414 on chromosome 5; (z) 7,810,719 on chromosome 5; (aa) 8,224,119 on chromosome 5; (bb) 9,503,718 on chromosome 5; (cc) 10,940,045 on chromosome 5; (dd) 10,995,445 on chromosome 5; (ee) 11,233,933 on chromosome 5; (ff) 11,252,809 on chromosome 5; (gg) 26,170,920 on chromosome 5; (hh) 72,427,511 on chromosome 5; (ii) 73,623,726 on chromosome 5; (jj) 74,208,470 on chromosome 5; (kk) 77,146,321 on chromosome 6; (ll) 77,468,821 on chromosome 6; (mm) 980,137 on chromosome X; or (nn) 984,754 on chromosome X, wherein the reference genome is the Abacus cannabis reference genome (version CsaAba2). In an embodiment, the nucleotide position comprises: (a) on chromosome 1: (1) a C/C genotype at position 15,287,266; or (2) an A/A genotype genotype at position 15,368,894, (b) on chromosome 2: (1) a G/G or G/A genotype at position 95,466,762; (2) an A/A or C/A genotype at position 119,379; (3) a C/C or T/C genotype at position 149,520; (4) an A/A or C/A genotype at position 181,346; (5) a G/G or G/C genotype at position 325,930; (6) an A/A or G/A genotype at position 396,437; (7) a T/T or T/C genotype at position 494,430; (8) an A/A or G/A genotype at position 887,683; (9) an A/A or C/A genotype at position 1,114,228; (10) a T/T or G/T genotype at position 1,262,671; (11) a C/C or C/A genotype at position 1,368,060; (12) an A/A or G/A genotype at position 1,430,121; (13) an A/A or G/A genotype at position 1,434,872; (14) an A/A or C/A genotype at position 1,489,951; (15) a C/C or C/A genotype at position 1,791,640; or (16) an A/A or G/A genotype at position 2,136,725; (c) on chromosome 4: (1) an A/A genotype at position 3,436,566; (2) a G/G genotype at position 3,502,761; (3) an A/A genotype at position 3,911,993; (4) a C/C genotype at position 3,946,006; (5) a G/G genotype at position 3,995,523; or (6) an A/A genotype at position 4,065,757; (d) on chromosome 5: (1) a G/G genotype at position 6,578,414; (2) a G/G genotype at position 7,810,719; (3) a C/C genotype at position 8,224,119; (4) a C/C genotype at position 9,503,718; (5) a C/C genotype at position 10,940,045; (6) a G/G genotype at position 10,995,445; (7) a G/G genotype at position 11,233,933; (8) a C/C genotype at position 11,252,809; (9) a C/C genotype at position 26,170,920]; (10) an A/A genotype at position 72,427,511; (11) a T/T genotype at position 73,623,726; or (12) an A/A genotype at position 74,208,470; (e) on chromosome 6: (1) a C/C genotype at position 77,146,321; or (2) a T/T genotype at position 77,468,821; (f) on chromosome X: (1) a G/G genotype at position 980,137; or (2) a T/T genotype at position 984,754; wherein the reference genome is the Abacus cannabis reference genome (version CsaAba2).

In an embodiment, the one or more markers comprises a polymorphism at position 26 of any one or more of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; or SEQ ID NO:40.

In an embodiment, the nucleotide position comprises: (1) a C/C genotype at position 26 of SEQ ID NO:1; (2) an A/A genotype at position 26 of SEQ ID NO:2; (3) a G/G or G/A genotype at position 26 of SEQ ID NO:3; (4) an A/A genotype at position 26 of SEQ ID NO:4; (5) a G/G genotype at position 26 of SEQ ID NO:5; (6) an A/A genotype at position 26 of SEQ ID NO:6; (7) a C/C genotype at position 26 of SEQ ID NO:7; (8) a G/G genotype at position 26 of SEQ ID NO:8; (9) an A/A genotype at position 26 of SEQ ID NO:9; (10) a G/G genotype at position 26 of SEQ ID NO:10; (11) a G/G genotype at position 26 of SEQ ID NO:11; (12) a C/C genotype at position 26 of SEQ ID NO:12; (13) a C/C genotype at position 26 of SEQ ID NO:13; (14) a C/C genotype at position 26 of SEQ ID NO:14; (15) a G/G genotype at position 26 of SEQ ID NO:15; (16) a G/G genotype at position 26 of SEQ ID NO:16; (17) a C/C genotype at position 26 of SEQ ID NO:17; (18) a C/C genotype at position 26 of SEQ ID NO:18; (19) an A/A genotype at position 26 of SEQ ID NO:19; (20) a T/T genotype at position 26 of SEQ ID NO:20; (21) an A/A genotype at position 26 of SEQ ID NO:21; (22) a C/C genotype at position 26 of SEQ ID NO:22; (23) a T/T genotype at position 26 of SEQ ID NO:23; (24) a G/G genotype at position 26 of SEQ ID NO:24; (25) a T/T genotype at position 26 of SEQ ID NO:25; (26) an A/A or C/A genotype at position 26 of SEQ ID NO:26; (27) a C/C or T/C genotype at position 26 of SEQ ID NO:27; (28) a A/A or C/A genotype at position 26 of SEQ ID NO:28; (29) a G/G or G/C genotype at position 26 of SEQ ID NO:29; (30) an A/A or G/A genotype at position 26 of SEQ ID NO:30; (31) a T/T or T/C genotype at position 26 of SEQ ID NO:31; (32) an A/A or G/A genotype at position 26 of SEQ ID NO:32; (33) an A/A or C/A genotype at position 26 of SEQ ID NO:33; (34) a T/T or G/T genotype at position 26 of SEQ ID NO:34; (35) a C/C or C/A genotype at position 26 of SEQ ID NO:35; (36) an A/A or G/A genotype at position 26 of SEQ ID NO:36; (37) an A/A or G/A genotype at position 26 of SEQ ID NO:37; (38) an A/A or C/A genotype at position 26 of SEQ ID NO:38; (39) a C/C or C/A genotype at position 26 of SEQ ID NO:39; (40) an A/A or G/A genotype at position 26 of SEQ ID NO:40; wherein the reference genome is the Abacus Cannabis reference genome (version CsaAba2).

In an embodiment, the one or more markers comprises a polymorphism relative to a reference genome within any one or more haplotypes wherein the haplotypes comprise the region: (a) on chromosome 1: (1) between positions 15,277,564 and 15,291,446; or (2) between positions 15,366,809 and 15,402,935; (b) on chromosome 2: (1) between positions 95,458,836 and 95,467,337; (2) between positions 87,222 and 161,432; (3) between positions 172,849 and 196,868; (4) between positions 294,611 and 388,264; (5) between positions 388,264 and 438,385; (6) between positions 438,385 and 511,858; (7) between positions 876,155 and 889,775; (8) between positions 1,088,510 and 1,124,989; (9) between positions 1,241,220 1,266,562; (10) between positions 1,357,068 and 1,373,756; (11) between positions 1,424,355 and 1,443,831; (12) between positions 1,489,815 and 1,506,952; (13) between positions 1,687,471 and 1,796,288; (14) between positions 2,003,496 and 2,236,802; or (c) on chromosome 4: (1) between positions 3,388,047 and 3,444,350; (2) between positions 3,495,075 and 3,515,283; (3) between positions 3,905,616 and 3,935,114; (4) between positions 3,937,348 and 3,954,523; (5) between positions 3,990,905 and 4,026,073; or (6) between positions 4,061,743 and 4,067,160; (d) on chromosome 5: (1) between positions 6,563,925 and 6,626,590; (2) between positions 7,801,902 and 7,837,200; (3) between positions 8,187,388 and 8,231,407; (4) between positions 9,496,232 and 9,507,458; (5) between positions 10,930,567 and 10,942,351; (6) between positions 10,944,510 and 11,018,166; (7) between positions 11,217,176 and 11,310,169; (8) between positions 26,164,885 and 26,184,130; (9) between positions 72,387,436 and 72,428,036; (10) between positions 73,620,411 and 73,629,456; (11) between positions 74,199,722 and 74,217,967; or (e) on chromosome 6: (1) between positions 77,135,699 and 77,152,519; or (2) between positions 77,362,431 and 77,488,204; (f) on chromosome X: (1) between positions 978,062 and 1,034,965, wherein the reference genome is the Abacus cannabis reference genome (version CsaAba2).

In an embodiment, the selecting comprises marker assisted selection. In an embodiment, the detecting comprises an oligonucleotide probe. In an embodiment, the method further comprises crossing the one or more plants comprising the indicated resistance to powdery mildew to produce one or more F1 or additional progeny plants, wherein at least one of the F1 or additional progeny plants comprises the indicated resistance to powdery mildew. In an embodiment, the crossing comprises selfing, sibling crossing, or backcrossing. In an embodiment, the at least one additional progeny plant comprising the indicated resistance to powdery mildew comprises an F2-F7 progeny plant. In an embodiment, the selfing, sibling crossing, or backcrossing comprises marker-assisted selection. In an embodiment, the selfing, sibling crossing, or backcrossing comprises marker-assisted selection for at least two generations. In an embodiment, the plant comprises a Cannabis plant.

In an embodiment, a method for selecting one or more plants comprising resistance to powdery mildew is provided, the method comprising replacing a nucleic acid sequence of a parent plant with a nucleic acid sequence conferring resistance to powdery mildew.

In an embodiment, a plant is selected by (i) obtaining nucleic acids from a sample cannabis plant or its germplasm; (ii) detecting one or more markers that indicate resistance to powdery mildew, and (iii) indicating resistance to powdery mildew, and in an embodiment the method further comprises selecting the one or more plants indicating resistance to powdery mildew. In an embodiment, a seed of the plant is provided. In an embodiment, a tissue culture of cells produced from the plant is provided. In an embodiment, a plant generated from the tissue culture is provided. In an embodiment, a protoplast produced from the plant is provided. In an embodiment, a method of generating a processed cannabis product comprising the use of the plant is provided. In an embodiment, a cannabis product produced from the plant is provided. In an embodiment, the product is a kief, hashish, bubble hash, an edible product, solvent reduced oil, sludge, e-juice, or tincture.

### BRIEF DESCRIPTION OF THE DRAWINGS

The skilled artisan will understand that the drawings, described below, are for illustration purposes only. The drawings are not intended to limit the scope of the present teachings in any way.

FIG. 1 illustrates the effect of a powdery mildew lesion.

### DETAILED DESCRIPTION OF THE INVENTION

These and other features of the present teachings will become more apparent from the description herein. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The present teachings relate generally to producing or developing Cannabis varieties having resistance to powdery mildew by selecting plants having markers indicating such resistance.

The terminology used in the disclosure herein is for the purpose of describing particular embodiments only and is not intended to be limiting of the disclosure. As used in the description of the embodiments of the disclosure and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, for example, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

### Definitions

The term “Abacus” as used herein refers to the Cannabis reference genome known as the Abacus reference genome (version CsaAba2).

The term “backcrossing” or “to backcross” refers to the crossing of an F1 hybrid with one of the original parents. A backcross is used to maintain the identity of one parent (species) and to incorporate a particular trait from a second parent (species). The best strategy is to cross the F1 hybrid back to the parent possessing the most desirable traits.

The term “beneficial” as used herein refers to an allele conferring resistance to powdery mildew.

The term “Cannabis” refers to plants of the genus Cannabis, including Cannabis sativa, Cannabis indica, and Cannabis ruderalis.

The term “cell” refers to a prokaryotic or eukaryotic cell, including plant cells, capable of replicating DNA, transcribing RNA, translating polypeptides, and secreting proteins.

The term “coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

The terms “construct,” “plasmid,” “vector,” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present invention. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

The term “cross”, “crossing”, “cross pollination” or “cross-breeding” refer to the process by which the pollen of one flower on one plant is applied (artificially or naturally) to the ovule (stigma) of a flower on another plant, or “selfing” where pollen from a plant is applied (artificially or naturally) to the ovule (stigma) of the same plant. Backcrossing is a process in which a breeder repeatedly crosses hybrid progeny, for example a first generation hybrid (F1), back to one of the parents of the hybrid progeny. Backcrossing can be used to introduce one or more single locus conversions from one genetic background into another.

The term “cultivar” means a group of similar plants that by structural features and performance (e.g., morphological and physiological characteristics) can be identified from other varieties within the same species. Furthermore, the term “cultivar” variously refers to a variety, strain or race of plant that has been produced by horticultural or agronomic techniques and is not normally found in wild populations. The terms cultivar, variety, strain, plant and race are often used interchangeably by plant breeders, agronomists and farmers.

The term “detect” or “detecting” refers to any of a variety of methods for determining the presence of a nucleic acid.

The term “expression” or “gene expression” relates to the process by which the coded information of a nucleic acid transcriptional unit (including, e.g., genomic DNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “functional” as used herein refers to DNA or amino acid sequences which are of sufficient size and sequence to have the desired function (i.e. the ability to cause expression of a gene resulting in gene activity expected of the gene found in a reference genome, e.g., the Abacus reference genome.)

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genetic modification” or “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

The term “genome” as it applies to plant cells encompasses not only chromosomal DNA found within the nucleus, but organelle DNA found within subcellular components (e.g., mitochondrial, plastid) of the cell.

The term “genotype” refers to the genetic makeup of an individual cell, cell culture, tissue, organism (e.g., a plant), or group of organisms at a particular locus.

The term “germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants can be grown, as well as plant parts, such as leafs, stems, pollen, or cells that can be cultured into a whole plant.

The term “haplotype” refers to the genotype of a plant at a plurality of genetic loci, e.g., a combination of alleles or markers. Haplotype can refer to sequence of polymorphisms at a particular locus, such as a single marker locus, or sequence polymorphisms at multiple loci along a chromosomal segment in a given genome. As used herein, a haplotype can be a nucleic acid region spanning two markers.

A plant is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

The term “hybrid” refers to a variety or cultivar that is the result of a cross of plants of two different varieties. A hybrid, as described here, can refer to plants that are genetically different at any particular loci number of loci. A hybrid can further include a plant that is a variety that has been bred to have at least one different characteristic from the parent. “F1 hybrid” refers to the first generation hybrid, “F2 hybrid” the second generation hybrid, “F3 hybrid” the third generation, and so on. A hybrid refers to any progeny that is either produced, or developed using research and development to create a new line having at least one distinct characteristic.

As used herein, the term “inbreeding” refers to the production of offspring via the mating between relatives. The plants resulting from the inbreeding process are referred to herein as “inbred plants” or “inbreds.”

The term “introduced” refers to a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

The term “isolated” as used herein means having been removed from its natural environment, or removed from other compounds present when the compound is first formed. The term “isolated” embraces materials isolated from natural sources as well as materials (e.g., nucleic acids and proteins) recovered after preparation by recombinant expression in a host cell, or chemically-synthesized compounds such as nucleic acid molecules, proteins, and peptides.

The term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (TO) plant regenerated from material of that line; (b) has a pedigree comprised of a TO plant of that line; or (c) is genetically very similar due to common ancestry (e.g., via inbreeding or selfing). In this context, the term “pedigree” denotes the lineage of a plant, e.g. in terms of the sexual crosses affected such that a gene or a combination of genes, in heterozygous (hemizygous) or homozygous condition, imparts a desired trait to the plant.

The term “marker,” “genetic marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide, and can be represented by one or more particular variant sequences, or by a consensus sequence. In another sense, a marker is an isolated variant or consensus of such a sequence. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Other examples of such markers are restriction fragment length polymorphism (RFLP) markers, amplified fragment length polymorphism (AFLP) markers, single nucleotide polymorphisms (SNPs), microsatellite markers (e.g. SSRs), sequence-characterized amplified region (SCAR) markers, cleaved amplified polymorphic sequence (CAPS) markers or isozyme markers or combinations of the markers described herein which defines a specific genetic and chromosomal location.

The term “marker assisted selection” refers to the diagnostic process of identifying, optionally followed by selecting a plant from a group of plants using the presence of a molecular marker as the diagnostic characteristic or selection criterion. The process usually involves detecting the presence of a certain nucleic acid sequence or polymorphism in the genome of a plant.

The term “powdery mildew” refers to a condition commonly found in plants, including Cannabis, caused by a fungal infection. The invention described herein is not limited to any specific fungus, and is meant to refer to any type of powdery mildew infection caused by any fungus.

The term “offspring” refers to any plant resulting as progeny from a vegetative or sexual reproduction from one or more parent plants or descendants thereof. For instance an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “percent sequence identity” or “percent identity” or “sequence identity” or “identity” are used interchangeably to refer to a sequence comparison based on identical matches between correspondingly identical positions in the sequences being compared between two or more amino acid or nucleotide sequences. The percent identity refers to the extent to which two optimally aligned polynucleotide or peptide sequences are invariant throughout a window of alignment of components, e.g., nucleotides or amino acids. Hybridization experiments and mathematical algorithms known in the art may be used to determine percent identity. Many mathematical algorithms exist as sequence alignment computer programs known in the art that calculate percent identity. These programs may be categorized as either global sequence alignment programs or local sequence alignment programs.

The term “plant” refers to a whole plant and any descendant, cell, tissue, or part of a plant. A class of plant that can be used in the present invention is generally as broad as the class of higher and lower plants amenable to mutagenesis including angiosperms (monocotyledonous and dicotyledonous plants), gymnosperms, ferns and multicellular algae. Thus, “plant” includes dicot and monocot plants. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks. Plant parts include harvestable parts and parts useful for propagation of progeny plants. Plant parts useful for propagation include, for example and without limitation: seed; fruit; a cutting; a seedling; a tuber; and a rootstock. A harvestable part of a plant may be any useful part of a plant, including, for example and without limitation: flower; pollen; seedling; tuber; leaf; stem; fruit; seed; and root. A plant cell is the structural and physiological unit of the plant, comprising a protoplast and a cell wall. A plant cell may be in the form of an isolated single cell, or an aggregate of cells (e.g., a friable callus and a cultured cell), and may be part of a higher organized unit (e.g., a plant tissue, plant organ, and plant). Thus, a plant cell may be a protoplast, a gamete producing cell, or a cell or collection of cells that can regenerate into a whole plant. As such, a seed, which comprises multiple plant cells and is capable of regenerating into a whole plant, is considered a “plant cell” in embodiments herein. In an embodiment described herein are plants in the genus of Cannabis and plants derived thereof, which can be produced asexual or sexual reproduction.

The term “plant part” or “plant tissue” refers to any part of a plant including but not limited to, an embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen. Plant part may also include certain extracts such as kief, oil, or hash which includes cannabis trichomes or glands.

The terms “polynucleotide,” “polynucleotide sequence,” “nucleotide,” “nucleotide sequence,” “nucleic acid sequence,” “nucleic acid fragment,” and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA comprises one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide. An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The term “polymorphism” refers to a difference in the nucleotide or amino acid sequence of a given region as compared to a nucleotide or amino acid sequence in a homologous-region of another individual, in particular, a difference in the nucleotide of amino acid sequence of a given region which differs between individuals of the same species. A polymorphism is generally defined in relation to a reference sequence. Polymorphisms include single nucleotide differences, differences in sequence of more than one nucleotide, and single or multiple nucleotide insertions, inversions and deletions; as well as single amino acid differences, differences in sequence of more than one amino acid, and single or multiple amino acid insertions, inversions, and deletions.

The term “probe” or “nucleic acid probe” or “oligonucleotide probe” as used herein, is defined to be a collection of one or more nucleic acid fragments whose specific hybridization to a nucleic acid sample comprising a region of interest can be detected. The probe may be unlabeled or labeled as described below so that its binding to the target nucleic acid of interest can be detected. What “probe” refers to specifically is clear from the context in which the word is used. The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill will recognize that the precise sequence of the particular probes described herein can be modified to a certain degree to produce probes that are “substantially identical” to the disclosed probes, but retain the ability to specifically bind to (i.e., hybridize specifically to) the same targets or samples as the probe from which they were derived (see discussion above). Such modifications are specifically covered by reference to the individual probes described herein.

The term “progeny” refers to any subsequent generation of a plant. Progeny is measured using the following nomenclature: F1 refers to the first generation progeny, F2 refers to the second generation progeny, F3 refers to the third generation progeny, and so on.

The term “promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

The term “protein” refers to amino acid polymers that contain at least five constituent amino acids that are covalently joined by peptide bonds. The constituent amino acids can be from the group of amino acids that are encoded by the genetic code, which include: alanine, valine, leucine, isoleucine, methionine, phenylalanine, tyrosine, tryptophan, serine, threonine, asparagine, glutamine, cysteine, glycine, proline, arginine, histidine, lysine, aspartic acid, and glutamic acid. As used herein, the term “protein” is synonymous with the related terms “peptide” and “polypeptide.”

The term “quantitative trait loci” or “QTL” refers to the genetic elements controlling a quantitative trait.

The term “reference plant” or “reference genome” refers to a wild-type or reference sequence that SNPs or other markers in a test sample can be compared to in order to detect a modification of the sequence in the test sample.

The phrase “resistance to powdery mildew” or “powdery mildew resistance” as used herein refers to the ability to inhibit or suppress the occurrence and progression of damage due to the infection with a powdery mildew pathogen. The resistance may mean any of the following, for example: to prevent the damage from occurring; to stop the progression of the damage that has occurred already; and to suppress or inhibit the progression of the damage that has occurred already.

The terms “similar,” “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. A “substantially homologous sequence” refers to variants of the disclosed sequences such as those that result from site-directed mutagenesis, as well as synthetically derived sequences. A substantially homologous sequence of the present invention also refers to those fragments of a particular promoter nucleotide sequence disclosed herein that operate to promote the constitutive expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present invention.

The term “single nucleotide polymorphism (SNP)” refers to a change in which a single base in the DNA differs from the usual base at that position. These single base changes are called SNPs or “snips.”

The term “target region” or “nucleic acid target” refers to a nucleotide sequence that resides at a specific chromosomal location. The “target region” or “nucleic acid target” is specifically recognized by a probe.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the said characteristics and iii) considered as a unit with regard to its suitability for being propagated unchanged.

Cannabis

Cannabis has long been used for drug and industrial purposes, fiber (hemp), for seed and seed oils, for medicinal purposes, and for recreational purposes. Industrial hemp products are made from Cannabis plants selected to produce an abundance of fiber. Some Cannabis varieties have been bred to produce minimal levels of THC, the principal psychoactive constituent responsible for the psychoactivity associated with marijuana. Marijuana has historically consisted of the dried flowers of Cannabis plants selectively bred to produce high levels of THC and other psychoactive cannabinoids. Various extracts including hashish and hash oil are also produced from the plant.

Cannabis is an annual, dioecious, flowering herb. The leaves are palmately compound or digitate, with serrate leaflets. Cannabis normally has imperfect flowers, with staminate “male” and pistillate “female” flowers occurring on separate plants. It is not unusual, however, for individual plants to separately bear both male and female flowers (i.e., have monoecious plants). Although monoecious plants are often referred to as “hermaphrodites,” true hermaphrodites (which are less common in Cannabis) bear staminate and pistillate structures on individual flowers, whereas monoecious plants bear male and female flowers at different locations on the same plant.

The life cycle of Cannabis varies with each variety but can be generally summarized into germination, vegetative growth, and reproductive stages. Because of heavy breeding and selection by humans, most Cannabis seeds have lost dormancy mechanisms and do not require any pre-treatments or winterization to induce germination (See Clarke, R C et al. “Cannabis: Evolution and Ethnobotany” University of California Press 2013). Seeds placed in viable growth conditions are expected to germinate in about 3 to 7 days. The first true leaves of a Cannabis plant contain a single leaflet, with subsequent leaves developing in opposite formation with increasing number of leaflets. Leaflets can be narrow or broad depending on the morphology of the plant grown. Cannabis plants are normally allowed to grow vegetatively for the first 4 to 8 weeks. During this period, the plant responds to increasing light with faster and faster growth. Under ideal conditions, Cannabis plants can grow up to 2.5 inches a day, and are capable of reaching heights of up to 20 feet. Indoor growth pruning techniques tend to limit Cannabis size through careful pruning of apical or side shoots.

Cannabis is diploid, having a chromosome complement of 2n=20, although polyploid individuals have been artificially produced. The first genome sequence of Cannabis, which is estimated to be 820 Mb in size, was published in 2011 by a team of Canadian scientists (Bakel et al, “The draft genome and transcriptome of Cannabis sativa” Genome Biology 12:R102).

All known varieties of Cannabis are wind-pollinated and the fruit is an achene. Most varieties of Cannabis are short day plants, with the possible exception of C. sativa subsp. sativa var. spontanea (=C. ruderalis), which is commonly described as “auto-flowering” and may be day-neutral.

The genus Cannabis was formerly placed in the Nettle (Urticaceae) or Mulberry (Moraceae) family, and later, along with the Humulus genus (hops), in a separate family, the Hemp family (Cannabaceae sensu stricto). Recent phylogenetic studies based on cpDNA restriction site analysis and gene sequencing strongly suggest that the Cannabaceae sensu stricto arose from within the former Celtidaceae family, and that the two families should be merged to form a single monophyletic family, the Cannabaceae sensu lato.

Cannabis plants produce a unique family of terpeno-phenolic compounds called cannabinoids. Cannabinoids, terpenoids, and other compounds are secreted by glandular trichomes that occur most abundantly on the floral calyxes and bracts of female plants. As a drug it usually comes in the form of dried flower buds (marijuana), resin (hashish), or various extracts collectively known as hashish oil. There are at least 483 identifiable chemical constituents known to exist in the Cannabis plant (Rudolf Brenneisen, 2007, Chemistry and Analysis of Phytocannabinoids (cannabinoids produced produced by Cannabis) and other Cannabis Constituents, In Marijuana and the Cannabinoids, ElSohly, ed.; incorporated herein by reference) and at least 85 different cannabinoids have been isolated from the plant (EI-Alfy, Abir T, et al., 2010, “Antidepressant-like effect of delta-9-tetrahydrocannabinol and other cannabinoids isolated from Cannabis sativa L”, Pharmacology Biochemistry and Behavior 95 (4): 434-42; incorporated herein by reference). The two cannabinoids usually produced in greatest abundance are cannabidiol (CBD) and/or A⁹-tetrahydrocannabinol (THC). THC is psychoactive while CBD is not. See, ElSohly, ed. (Marijuana and the Cannabinoids, Humana Press Inc., 321 papers, 2007), which is incorporated herein by reference in its entirety, for a detailed description and literature review on the cannabinoids found in marijuana.

Cannabinoids are the most studied group of secondary metabolites in Cannabis. Most exist in two forms, as acids and in neutral (decarboxylated) forms. The acid form is designated by an “A” at the end of its acronym (i.e. THCA). The phytocannabinoids are synthesized in the plant as acid forms, and while some decarboxylation does occur in the plant, it increases significantly post-harvest and the kinetics increase at high temperatures. (Sanchez and Verpoorte 2008). The biologically active forms for human consumption are the neutral forms. Decarboxylation is usually achieved by thorough drying of the plant material followed by heating it, often by either combustion, vaporization, or heating or baking in an oven. Unless otherwise noted, references to cannabinoids in a plant include both the acidic and decarboxylated versions (e.g., CBD and CBDA).

Detection of neutral and acidic forms of cannabinoids are dependent on the detection method utilized. Two popular detection methods are high-performance liquid chromatography (HPLC) and gas chromatography (GC). HPLC separates, identifies, and quantifies different components in a mixture, and passes a pressurized liquid solvent containing the sample mixture through a column filled with a solid adsorbent material. Each molecular component in a sample mixture interacts differentially with the adsorbent material, thus causing different flow rates for the different components and therefore leading to separation of the components. In contrast, GC separates components of a sample through vaporization. The vaporization required for such separation occurs at high temperature. Thus, the main difference between GC and HPLC is that GC involves thermal stress and mainly resolves analytes by boiling points while HPLC does not involve heat and mainly resolves analytes by polarity. The consequence of utilizing different methods for cannabinoid detection therefore is that HPLC is more likely to detect acidic cannabinoid precursors, whereas GC is more likely to detect decarboxylated neutral cannabinoids.

The cannabinoids in cannabis plants include, but are not limited to, Δ9-Tetrahydrocannabinol (Δ9-THC), Δ8-Tetrahydrocannabinol (Δ8-THC), Cannabichromene (CBC), Cannabicyclol (CBL), Cannabidiol (CBD), Cannabielsoin (CBE), Cannabigerol (CBG), Cannabinidiol (CBND), Cannabinol (CBN), Cannabitriol (CBT), and their propyl homologs, including, but are not limited to cannabidivarin (CBDV), Δ9-Tetrahydrocannabivarin (THCV), cannabichromevarin (CBCV), and cannabigerovarin (CBGV). See Holley et al. (Constituents of Cannabis sativa L. XI Cannabidiol and cannabichromene in samples of known geographical origin, J. Pharm. Sci. 64:892-894, 1975) and De Zeeuw et al. (Cannabinoids with a propyl side chain in Cannabis, Occurrence and chromatographic behavior, Science 175:778-779), each of which is herein incorporated by reference in its entirety for all purposes. Non-THC cannabinoids can be collectively referred to as “CBs”, wherein CBs can be one of THCV, CBDV, CBGV, CBCV, CBD, CBC, CBE, CBG, CBN, CBND, and CBT cannabinoids.

### Markers and Haplotypes

The present invention describes the discovery of novel markers indicating resistance to powdery mildew, the method comprising i) obtaining nucleic acids from a sample plant or its germplasm; (ii) detecting one or more markers that indicate resistance to powdery mildew, and (iii) indicating resistance to powdery mildew. An embodiment further describes selecting the one or more plants indicating resistance to powdery mildew.

The markers of the present invention were discovered as described herein, which comprise polymorphisms relative to the Abacus cannabis reference genome (version CsaAba2). In an embodiment, as described in Tables 1 and 2, the markers identify polymorphisms that indicate resistance to powdery mildew. Tables 1 and 2 describe the markers and sequence identifiers, and the positioning on their respective chromosomes. Tables 1 and 2 further describe the reference call of the nucleotide at the respective position within the reference genome, as well as the alternate call describing the polymorphism in plants having resistance to powdery mildew. Tables 1 and 2 also describe the beneficial genotype with respect to the described markers.

The markers of the present invention may be described in numerous fashions. To illustrate, for non-limiting exemplary purposes, marker 157_2302749 found in Table 1 is described as being positioned at base pair (bp) position 15,287,266 on chromosome 1 of the CsaAba2 reference genome. Likewise, marker 157_2302749 is described as being positioned at nucleotide 26 of SEQ ID NO:1.

The present invention further describes the discovery of novel haplotype markers for plants, including cannabis. Haplotypes refer to the genotype of a plant at a plurality of genetic loci, e.g., a combination of alleles or markers. Haplotypes can refer to sequence polymorphisms at a particular locus, such as a single marker locus, or sequence polymorphisms at multiple loci along a chromosomal segment in a given genome. Markers of the present invention and within the haplotypes described are significantly correlated to plants having resistance to powdery mildew, which thus can be used to screen plants exhibiting resistance to powdery mildew.

In an embodiment, Tables 1 and 2 describe markers within a haplotype that identify polymorphisms that confer resistance to powdery mildew. In particular, Tables 1 and 2 describe the haplotype both with respect to the left and right flanking markers, and with respect to the left and right flanking positioning on their respective chromosomes. To illustrate, for non-limiting exemplary purposes, marker 157_2302749 is within a haplotype defined as being between left flanking marker 157_2293833 at position 15,277,564 on chromosome 1 and right flanking marker 157_2306929 at position 15,291,446 on chromosome 1 of the CsaAba2 reference genome.

### Quantitative Trait Loci

The term chromosome interval designates a contiguous linear span of genomic DNA that resides on a single chromosome. A chromosome interval may comprise a quantitative trait locus (“QTL”) linked with a genetic trait and the QTL may comprise a single gene or multiple genes associated with the genetic trait. The boundaries of a chromosome interval comprising a QTL are drawn such that a marker that lies within the chromosome interval can be used as a marker for the genetic trait, as well as markers genetically linked thereto. Each interval comprising a QTL comprises at least one gene conferring a given trait, however knowledge of how many genes are in a particular interval is not necessary to make or practice the invention, as such an interval will segregate at meiosis as a linkage block. In accordance with the invention, a chromosomal interval comprising a QTL may therefore be readily introgressed and tracked in a given genetic background using the methods and compositions provided herein.

Identification of chromosomal intervals and QTL is therefore beneficial for detecting and tracking a genetic trait, such as resistance to powdery mildew, in plant populations. In some embodiments, this is accomplished by identification of markers linked to a particular QTL. The principles of QTL analysis and statistical methods for calculating linkage between markers and useful QTL include penalized regression analysis, ridge regression, single point marker analysis, complex pedigree analysis, Bayesian MCMC, identity-by-descent analysis, interval mapping, composite interval mapping (CIM), and Haseman-Elston regression. QTL analyses may be performed with the help of a computer and specialized software available from a variety of public and commercial sources known to those of skill in the art.

### Detection of Markers

The present invention describes the use of detecting markers associated with resistance to powdery mildew. Marker detection is well known in the art. For example, amplification of a target polynucleotide (e.g., by PCR) using a particular amplification primer pair that permit the primer pair to hybridize to the target polynucleotide to which a primer having the corresponding sequence (or its complement) would bind and preferably to produce an identifiable amplification product (the amplicon) having a marker is well known in the art.

Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Methods of amplification are further described in U.S. Pat. Nos. 4,683,195, 4,683,202 and Chen et al. (1994) PNAS 91:5695-5699. These methods as well as other methods known in the art of DNA amplification may be used in the practice of the embodiments of the present invention. It will be appreciated that suitable primers to be used with the invention can be designed using any suitable method. It is not intended that the invention be limited to any particular primer or primer pair. It is not intended that the primers of the invention be limited to generating an amplicon of any particular size. For example, the primers used to amplify the marker loci and alleles herein are not limited to amplifying the entire region of the relevant locus. The primers can generate an amplicon of any suitable length that is longer or shorter than those disclosed herein. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. It is understood that a number of parameters in a specific PCR protocol may need to be adjusted to specific laboratory conditions and may be slightly modified and yet allow for the collection of similar results. The primers of the invention may be radiolabeled, or labeled by any suitable means (e.g., using a non-radioactive fluorescent tag), to allow for rapid visualization of the different size amplicons following an amplification reaction without any additional labeling step or visualization step. The known nucleic acid sequences for the genes described herein are sufficient to enable one of skill in the art to routinely select primers for amplification of the gene of interest.

Other suitable amplification methods include, but are not limited to, ligase chain reaction (LCR) (see, Wu and Wallace (1989) Genomics 4: 560, Landegren et al. (1988) Science 241: 1077, and Barringer et al. (1990) Gene 89: 117), transcription amplification (Kwoh et al. (1989) Proc. Natl. Acad. Sci. USA 86: 1173), self-sustained sequence replication (Guatelli et al. (1990) Proc. Natl. Acad. Sci. USA 87: 1874), dot PCR, and linker adapter PCR, etc.

An amplicon is an amplified nucleic acid, e.g., a nucleic acid that is produced by amplifying a template nucleic acid by any available amplification method (e.g., PCR, LCR, transcription, or the like). A genomic nucleic acid is a nucleic acid that corresponds in sequence to a heritable nucleic acid in a cell. Common examples include nuclear genomic DNA and amplicons thereof. A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A template nucleic acid is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST. Details regarding the use of these and other amplification methods can be found in any of a variety of standard texts. Many available biology texts also have extended discussions regarding PCR and related amplification methods and one of skill will appreciate that essentially any RNA can be converted into a double stranded DNA suitable for restriction digestion, PCR expansion and sequencing using reverse transcriptase and a polymerase.

PCR detection and quantification using dual-labeled fluorogenic oligonucleotide probes, commonly referred to as “TaqMan™” probes, can also be performed according to the present invention. These probes are composed of short (e.g., 20-25 base) oligodeoxynucleotides that are labeled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. TaqMan™ probes are oligonucleotides that have a label and a quencher, where the label is released during amplification by the exonuclease action of the polymerase used in amplification, providing a real time measure of amplification during synthesis. A variety of TaqMan™ reagents are commercially available, e.g., from Applied Biosystems as well as from a variety of specialty vendors such as Biosearch Technologies.

In general, synthetic methods for making oligonucleotides, including probes, primers, molecular beacons, PNAs, LNAs (locked nucleic acids), etc., are well known. For example, oligonucleotides can be synthesized chemically according to the solid phase phosphoramidite triester method described. Oligonucleotides, including modified oligonucleotides, can also be ordered from a variety of commercial sources.

Nucleic acid probes to the marker loci can be cloned and/or synthesized. Any suitable label can be used with a probe of the invention. Detectable labels suitable for use with nucleic acid probes include, for example, any composition detectable by spectroscopic, radioisotopic, photochemical, biochemical, immunochemical, electrical, optical or chemical means. Useful labels include biotin for staining with labeled streptavidin conjugate, magnetic beads, fluorescent dyes, radio labels, enzymes, and colorimetric labels. Other labels include ligands which bind to antibodies labeled with fluorophores, chemiluminescent agents, and enzymes. A probe can also constitute radio labeled PCR primers that are used to generate a radio labeled amplicon. It is not intended that the nucleic acid probes of the invention be limited to any particular size.

Amplification is not always a requirement for marker detection (e.g. Southern blotting and RFLP detection). Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

### Powdery Mildew Resistance Genes

In an embodiment, candidate genes conferring resistance to powdery mildew are provided. As described herein, candidate genes for powdery mildew resistance may include, but are not limited to, putative recombination initiation defects 3 (PRD3; AT1G01690; meiotic double strand break formation): Cytochrome P450 86A8 (CYP86A8; At2g45970; biosynthesis of lipids for cuticle, affects cuticle permeability, resistance to the fungal pathogen Botrytis cinerea (Bessire, Michael, et al. “A permeable cuticle in Arabidopsis leads to a strong resistance to Botrytis cinerea.” The EMBO journal 26.8 (2007): 2158-2168.)); a copia-like retrotransposable element; probable indole-3-pyruvate monooxygenase YUCCA11 (YUC11; At1g21430; Involved in auxin biosynthesis); queuine tRNA-ribosyltransferase catalytic subunit 1 isoform X1 (Cannabis sativa, no Arabidopsis homolog was found); calcium-dependent protein kinase 1/4/10/14/23/30 (CPK1/4/10/14/23/30; calcium-dependent protein kinase; CPK23 is involved in stress response (Shi, Sujuan, et al. “The Arabidopsis calcium-dependent protein kinases (CDPKs) and their roles in plant growth regulation and abiotic stress responses.” International journal of molecular sciences 19.7 (2018): 1900.); embryonic stem cell-specific 5-hydroxymethylcytosine-binding protein (AT2G26470), hypothetical protein AT3G07440, HAUS augmin-like complex subunit (AUG3; AT5G48520; microtubule organization during plant cell division); pathogenic type III effector avirulence factor Avr AvrRpt-cleavage: cleavage site protein (AT5G48500); putative lipid-transfer protein (DIR1; At5g48485; Putative lipid transfer protein required for systemic acquired resistance (SAR) long distance signaling (Maldonado, Ana M., et al. “A putative lipid transfer protein involved in systemic resistance signalling in Arabidopsis” Nature 419.6905 (2002): 399-403)); Fimbrin-1/2/3/5/5 (FIM1/2/3/4/5; AT5G48460; actin bundle development); NTP2 (AT2G40520; unknown function); Chaperone protein dnaJ 11, chloroplastic (J11/DJC23; AT4G36040; unknown function); DNA-(apurinic or apyrimidinic site) endonuclease 2 (APE2; At4g36050); basic Helix-Loop-Helix 121 (BHLH121; At3g19860; transcription factor); Calcium-dependent protein kinase 16 (CPK16; AT1G17890; Calcium Dependent Protein Kinase); Protein kinase superfamily protein (AT1G65950), Naringenin,2-oxoglutarate 3-dioxygenase (F3H; At3g51240; flavonoid biosynthesis), Chalcone-flavanone isomerase family protein (AT5G66230), hypothetical protein AT2G13240; RNA demethylase ALKBH9B (ALKBH9B; At2g17970; Dioxygenase that demethylates RNA); G-type lectin S-receptor-like serine/threonine-protein kinase (At5g24080); Tic22-like family protein (AT5G62650) WD repeat-containing protein ATCSA-1(AT1G27840; UV-B tolerance and genome integrity); DUF21 domain-containing protein (CBSDUF6; At4g33700); Polygalacturonase Clade F 3 (PGF3; AT2G23900); hypothetical protein AT3G07750 and CYP715A1 (AT5G51280); snc1-influencing plant E3 ligase reverse genetic screen 4 (SNIPER4; AT3G48880; involved in plant immunity (Huang, Jianhua, Chipan Zhu, and Xin Li. “SCFSNIPER4 controls the turnover of two redundant TRAF proteins in plant immunity,” The Plant Journal 95.3 (2018): 504-515.)); LOC115717563 (located between 12,046,739-12,047,898 bp on chromosome 5 of the CBDRx reference genome; uncharacterized protein); LOC115717014 (located between 12,052,783-12,055,690 bp on chromosome 5 of the CBDRx reference genome; cytokinin hydroxylase-like), LOC115715834 (located between 12,078,573-12,081,614 bp on chromosome 5 of the CBDRx reference genome); LOC115717564 (located between 12,084,431 . . . 12,085,162 bp on chromosome 5 of the CBDRx reference genome; uncharacterized protein); LOC115717565 (located between 12,096,843-12,097,511 bp of the CBDRx reference genome; uncharacterized protein); LOC115716248 (tripeptidyl-peptidase 2-like); Phosphate transporter PHO1 homolog 10 (PHO1-H10; At1g69480); Carotenoid cleavage dioxygenase 7, chloroplastic (CCD7; At2g44990); SBP (S-ribonuclease binding protein) family protein (AT1G32740); LOC115718090 (located between positions 83,054,167-83,055,104 on chromosome 5 of the CBDRx reference genome; uncharacterized protein); LOC115718091 (located between positions 83,067,023-83,068,240 on chromosome 5 of the CBDRx reference genome; uncharacterized protein); hypothetical protein AT4G28025; tRNA-thr(GGU) m(6)t(6)A37 methyltransferase (AT4G28020); Xyloglucan endotransglucosylase/hydrolase protein 14 (XTH14; AT4G25820), Xyloglucan endotransglucosylase/hydrolase protein 15 (XTH15; AT4G14130); Diacylglycerol kinase 3/4/7 (DGK3/4/7; At4g30340; Phosphorylation of diacylglycerol to generate phosphatidic acid, which is required for response to pathogen attack (Arisz, Steven A., et al. “Rapid phosphatidic acid accumulation in response to low temperature stress in Arabidopsis is generated through diacylglycerol kinase.” Frontiers in plant science 4 (2013): 1.)); Acetyl-CoA carboxylase 1 (ACC1; At1g36160; fatty acid biosynthesis, cuticle permeability (Monda, Keina, et al. “Cuticle permeability is an important parameter for the trade-off strategy between drought tolerance and C02 uptake in land plants.” Plant Signaling & Behavior 16.6 (2021): 1908692.)); Wax ester synthase/diacylglycerol acyltransferase 1 (WSD1; At5g37300; involved in cuticular wax biosynthesis; Li, Fengling, et al. “Identification of the wax ester synthase/acyl-coenzyme A: diacylglycerol acyltransferase WSD1 required for stem wax ester biosynthesis in Arabidopsis.” Plant physiology 148.1 (2008): 97-107); RNA-binding (RRM/RBD/RNP motifs) family protein (AT5G66010, Mannose-P-dolichol utilization defect 1 protein homolog 1 (At5g59470), Transducin/WD40 repeat-like superfamily protein (AT4G14310); Transcription initiation factor TFIID subunit 15 (TAF15; At1g50300; essential for mediating regulation of RNA polymerase transcription), Hexokinase-4 (At3g20040; Fructose and glucose phosphorylating enzyme); Putative pentatricopeptide repeat-containing protein (PCMP-E82; At3g05240; mitochondrial mRNA modification); UDP-Glycosyltransferase superfamily protein (AT5G12890), UDP-glycosyltransferase 92A1 (UGT92A1; AT5G12890); UDP-glycosyltransferase 92A1 (UGT92A1; AT5G12890); Lysine-specific demethylase (JMJ30; At3g20810; Involved in the control of flowering time by demethylating H3K36me2 at the FT locus and repressing its expression): ETHYLENE INSENSITIVE 3-like 3 protein (EIL3: AT1G73730; Probable transcription factor that may be involved in the ethylene response pathway); 4-coumarate-CoA ligase-like protein (At1g20480; CoA-ligase activity); FT-interacting protein 1 (FTIP1; At5g06850; regulates flowering time under long days); NAD(P)-binding Rossmann-fold superfamily protein (AT3G20790; NADPH regeneration); Lipoxygenase 6, chloroplastic (LOX6; At1g67560; may be involved in pest resistance (Bell, Erin, Robert A. Creelman, and John E Mullet. “A chloroplast lipoxygenase is required for wound-induced jasmonic acid accumulation in Arabidopsis.” Proceedings of the National Academy of Sciences 92.19 (1995): 8675-8679); Linoleate 9S-lipoxygenase 1 (LOX1; At1g55020; defense response (Marcos, Ruth, et al. “9-Lipoxygenase-derived oxylipins activate brassinosteroid signaling to promote cell wall-based defense and limit pathogen infection.” Plant physiology 169.3 (2015): 2324-2334.); Lipoxygenase 2, chloroplastic (LOX2; At3g45140; may be involved in pest resistance (Bell, Erin, Robert A. Creelman, and John E. Mullet. “A chloroplast lipoxygenase is required for wound-induced jasmonic acid accumulation in Arabidopsis.” Proceedings of the National Academy of Sciences 92.19 (1995): 8675-8679)); beta glucosidase 15 (BGLU15; AT2G44450; beta-glucosidase activity); beta glucosidase 15 (BGLU15; AT2G44450; beta-glucosidase activity); beta glucosidase 17 (BGLU17; AT2G42040; transcription cis-regulatory region binding to AT5G44030 (Taylor-Teeples, Mallory, et al. “An Arabidopsis gene regulatory network for secondary cell wall synthesis.” Nature 517.7536 (2015): 571-575.)); a cellulose synthase involved in secondary cell wall biosynthesis and bacterial and fungal pathogen resistance (Hernández-Blanco, Camilo, et al. “Impairment of cellulose synthases required for Arabidopsis secondary cell wall formation enhances disease resistance.” The Plant Cell 19.3 (2007): 890-903.)); rho GTPase-activating gacO-like protein (AT3G57930; defense response inferred from genomics data (Depuydt, Thomas, and Klaas Vandepoele. “Multi-omics network-based functional annotation of unknown Arabidopsis genes.” bioRxiv (2021).); hypothetical protein (AT5G59020); CEL-Activated Resistance 1 (CAR1; AT1G50180; immune receptor which recognizes the conserved effectors AvrE and HopAA1 (Laflamme, Bradley, et al. “The pan-genome effector-triggered immunity landscape of a host-pathogen interaction.” Science 367.6479 (2020): 763-768.)); Disease resistance protein (CC-NBS-LRR class) family (AT1G53350); receptor-like protein kinase 1 (RLK1; AT5G60900; defense response to fungus (Brotman, Yariv, et al. “The LysM receptor-like kinase LysM RLK1 is required to activate defense and abiotic-stress responses induced by overexpression of fungal chitinases in Arabidopsis plants.” Molecular plant 5.5 (2012): 1113-1124.)); receptor-like protein kinase 1 (RLK1; AT5G60900; defense response to fungus (Brotman, Yariv, et al. “The LysM receptor-like kinase LysM RLK1 is required to activate defense and abiotic-stress responses induced by overexpression of fungal chitinases in Arabidopsis plants.” Molecular plant 5.5 (2012): 1113-1124.); CEL-Activated Resistance 1 (CAR1; AT1G50180; immune receptor which recognizes the conserved effectors AvrE and HopAA1 (Laflamme, Bradley, et al. “The pan-genome effector-triggered immunity landscape of a host-pathogen interaction.” Science 367.6479 (2020): 763-768.)); Disease resistance protein (CC-NBS-LRR class) family (AT1G53350); Disease resistance protein (CC-NBS-LRR class) family (AT1G53350); Cysteine—tRNA ligase 2, cytoplasmic (At5g38830), receptor-like protein kinase 1 (RLK1; AT5G60900; defense response to fungus (Brotman, Yariv, et al. “The LysM receptor-like kinase LysM RLK1 is required to activate defense and abiotic-stress responses induced by overexpression of fungal chitinases in Arabidopsis plants.” Molecular plant 5.5 (2012): 1113-1124.)); Cation efflux family protein (MTP11; AT2G39450; manganese transporter); Delta(12)-fatty-acid desaturase (FAD2; At3g12120; fatty acid biosynthesis, resistance to fungus resulting from cuticle permeability alterations (Dubey, Olga, et al. “Plant surface metabolites as potent antifungal agents.” Plant Physiology and Biochemistry 150 (2020): 39-48.)), anaphase-promoting complex subunit 8 (APC8; AT3G48150); Probable ion channel POLLUX (At5g49960); F-box and associated interaction domains-containing protein (AT3G17570), Protein DMP3 (At4g24310; membrane remodelling); DNA-binding bromodomain-containing protein (AT1G58025); Pseudouridine synthase family protein (AT1G09800); ARM repeat superfamily protein (AT3G03440; defense response to bacterium inferred from genomics data (Depuydt, Thomas, and Klaas Vandepoele. “Multi-omics network-based functional annotation of unknown Arabidopsis genes.” bioRxiv (2021).): resistance protein Ler3 (At5g48620; defense response inferred from genomics data (Depuydt, Thomas, and Klaas Vandepoele. “Multi-omics network-based functional annotation of unknown Arabidopsis genes.” bioRxiv (2021)); nuclease (AT5G41980); CCR4-NOT transcription complex subunit (AT5G18420); Protein kinase superfamily protein (AT2G40980); RINGiU-box superfamily protein (AT1G47570); ABC transporter D family member 1 (ABCD1; At4g39850; lipid catabolic process); TOM1-LIKE 5 (TOL5; AT5G63640; ubiquitin binding protein); protection of telomeres 1b (POT1b; AT5G06310; telomere capping); RNA-binding KH domain-containing protein (AT1G09660); Cytokinin riboside 5-monophosphate phosphoribohydrolase (LOG5; At4g35190; Cytokinin-activating enzyme), pollen receptor like kinase 3 (PRK3; AT3G42880); glycosyltransferase family protein 2 (AT5G60700); trimethylguanosine synthase (AT2G28310); UDP-xylose transporter 3 (UXT3; At1g06890; nucleotide-sugar transporter); Pieckstrin homology (PH) and lipid-binding START domains-containing protein (AT2G28320; contains region with similarity to EDR2, which is involved in powdery mildew resistance (Vorwerk, Sonja, et al. “EDR2 negatively regulates salicylic acid-based defenses and cell death during powdery mildew infections of Arabidopsis thaliana.” BMC plant biology 7.1 (2007): 1-14.)); spore wall protein 2-like, partial (Cannabis sativa, no significant homology with Arabidopsis thaliana); Protein-tyrosine sulfotransferase (TPST; At1g08030; innate immune response (lgarashi, Daisuke, Kenichi Tsuda, and Fumiaki Katagiri. “The peptide growth factor, phytosulfokine, attenuates pattern-triggered immunity.” The plant journal 71.2 (2012): 194-204.)); GATA transcription factor 10 (GATA10; AT1G08000; zinc finger transcription factor); transmembrane protein (AT2G28410), zinc finger, C3HC4 type family protein (AT2G28430); zinc finger, C3HC4 type family protein (AT2G28430); zinc finger, C3HC4 type family protein (AT2G28430); zinc finger, C3HC4 type family protein (AT2G28430); zinc finger, C3HC4 type family protein (AT2G28430); zinc finger, C3HC4 type family protein (AT2G28430); zinc finger, C3HC4 type family protein (AT2G28430); C3HC4 type family protein (AT2G28430); Protein disulfide isomerase-like 1-4 (PDIL1-4; At5g60640; protein folding); APAP1 (AT3G39080); 4-hydroxy-3-methylbut-2-en-1-yl diphosphate synthase (ferredoxin); chloroplastic (ISPG; At5g60600; involved in systemic acquired resistance (Gil, Ma José, et al. “The Arabidopsis csb3 mutant reveals a regulatory link between salicylic acid-mediated disease resistance and the methyl-erythritol 4-phosphate pathway.” The Plant Journal 44.1 (2005): 155-166.)), DHBP synthase RibB-like alpha/beta domain-containing protein (AT5G60590); RING/U-box superfamily protein (AT5g60580); Zinc finger CCCH domain-containing protein 24 (At2g23450); Probable E3 ubiquitin-protein ligase (LOG2; At3g09770); F-box/kelch-repeat protein (At5g60570); Protein DMP7 (At4g28485; abscission); Serine/threonine-protein kinase GRIK1 (At3g45240; Activates SnRK1.1/KIN10 and SnRK1.2/KIN11 by phosphorylation).

Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 72% to 100%, such as 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

In an embodiment, an isolated polynucleotide is provided comprising a nucleotide sequence having at least 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100% sequence identity compared to the claimed sequence, based on the Clustal V method of alignment with pairwise alignment default parameters (KTUPLE=2, GAP PENALTY=5, WINDOW=4 and DIAGONALS SAVED=4).

Local sequence alignment programs are similar in their calculation, but only compare aligned fragments of the sequences rather than utilizing an end-to-end analysis. Local sequence alignment programs such as BLAST can be used to compare specific regions of two sequences. A BLAST comparison of two sequences results in an E-value, or expectation value, that represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GENBANK, have generally increased over time for any given query/entry match. In setting criteria for confidence of polypeptide function prediction, a “high” BLAST match is considered herein as having an E-value for the top BLAST hit of less than 1E-30; a medium BLASTX E-value is 1E-30 to 1E-8; and a low BLASTX E-value is greater than 1E-8. The protein function assignment in the present invention is determined using combinations of E-values, percent identity, query coverage and hit coverage. Query coverage refers to the percent of the query sequence that is represented in the BLAST alignment. Hit coverage refers to the percent of the database entry that is represented in the BLAST alignment. In one embodiment of the invention, function of a query polypeptide is inferred from function of a protein homolog where either (1) hit_p<1e-30 or % identity >35% AND query_coverage >50% AND hit_coverage >50%, or (2) hit_p<1e-8 AND query_coverage >70% AND hit_coverage >70%. The following abbreviations are produced during a BLAST analysis of a sequence. SEQ_NUM provides the SEQ ID NO for the listed recombinant polynucleotide sequences. CONTIG_ID provides an arbitrary sequence name taken from the name of the clone from which the cDNA sequence was obtained. PROTEIN_NUM provides the SEQ ID NO for the recombinant polypeptide sequence NCBI_GI provides the GenBank ID number for the top BLAST hit for the sequence. The top BLAST hit is indicated by the National Center for Biotechnology Information GenBank Identifier number. NCBI_GI_DESCRIPTION refers to the description of the GenBank top BLAST hit for sequence. E_VALUE provides the expectation value for the top BLAST match. MATCH_LENGTH provides the length of the sequence which is aligned in the top BLAST match TOP_HIT_PCT_IDENT refers to the percentage of identically matched nucleotides (or residues) that exist along the length of that portion of the sequences which is aligned in the top BLAST match. CAT_TYPE indicates the classification scheme used to classify the sequence. GO_BP=Gene Ontology Consortium—biological process; GO_CC=Gene Ontology Consortium—cellular component; GO_MF=Gene Ontology Consortium molecular function; KEGG=KEGG functional hierarchy (KEGG=Kyoto Encyclopedia of Genes and Genomes); EC=Enzyme Classification from ENZYME data bank release 25.0; POI=Pathways of Interest. CAT_DESC provides the classification scheme subcategory to which the query sequence was assigned. PRODUCT_CAT_DESC provides the FunCAT annotation category to which the query sequence was assigned. PRODUCT_HIT_DESC provides the description of the BLAST hit which resulted in assignment of the sequence to the function category provided in the cat_desc column. HIT_E provides the E value for the BLAST hit in the hit_desc column. PCT_IDENT refers to the percentage of identically matched nucleotides (or residues) that exist along the length of that portion of the sequences which is aligned in the BLAST match provided in hit_desc. QRY_RANGE lists the range of the query sequence aligned with the hit. HIT_RANGE lists the range of the hit sequence aligned with the query. provides the percent of query sequence length that matches QRY_CVRG provides the percent of query sequence length that matches to the hit (NCBI) sequence in the BLAST match (% qry cvrg=(match length/query total length)×100). HIT_CVRG provides the percent of hit sequence length that matches to the query sequence in the match generated using BLAST (% hit cvrg=(match lengthy hit total length)×100).

Methods for aligning sequences for comparison are well-known in the art. Various programs and alignment algorithms are described. In an embodiment, the subject disclosure relates to calculating percent identity between two polynucleotides or amino acid sequences using an AlignX alignment program of the Vector NTI suite (Invitrogen, Carlsbad, Calif.). The AlignX alignment program is a global sequence alignment program for polynucleotides or proteins. In an embodiment, the subject disclosure relates to calculating percent identity between two polynucleotides or amino acid sequences using the MegAlign program of the LASERGENE bioinformatics computing suite (MegAlign™ (.COPYRGT.1993-2016). DNASTAR. Madison, Wis.). The MegAlign program is a global sequence alignment program for polynucleotides or proteins.

### Cannabis Breeding

Cannabis is an important and valuable crop. Thus, a continuing goal of Cannabis plant breeders is to develop stable, high yielding Cannabis cultivars that are agronomically sound. To accomplish this goal, the Cannabis breeder preferably selects and develops Cannabis plants with traits that result in superior cultivars. The plants described herein can be used to produce new plant varieties. In some embodiments, the plants are used to develop new, unique, and superior varieties or hybrids with desired phenotypes.

The development of commercial Cannabis cultivars requires the development of Cannabis varieties, the crossing of these varieties, and the evaluation of the crosses. Pedigree breeding and recurrent selection breeding methods may be used to develop cultivars from breeding populations. Breeding programs may combine desirable traits from two or more varieties or various broad-based sources into breeding pools from which cultivars are developed by selfing and selection of desired phenotypes. The new cultivars may be crossed with other varieties and the hybrids from these crosses are evaluated to determine which have commercial potential.

Details of existing Cannabis plants varieties and breeding methods are described in Potter et al. (2011, World Wide Weed: Global Trends in Cannabis Cultivation and Its Control), Holland (2010, The Pot Book: A Complete Guide to Cannabis, Inner Traditions/Bear & Co, ISBN1594778981, 9781594778988), Green I (2009, The Cannabis Grow Bible: The Definitive Guide to Growing Marijuana for Recreational and Medical Use, Green Candy Press, 2009, ISBN 1931160589, 9781931160582), Green II (2005, The Cannabis Breeder's Bible: The Definitive Guide to Marijuana Genetics, Cannabis Botany and Creating Strains for the Seed Market, Green Candy Press, 1931160279, 9781931160278), Starks (1990, Marijuana Chemistry: Genetics, Processing & Potency, ISBN 0914171399, 9780914171393), Clarke (1981, Marijuana Botany, an Advanced Study: The Propagation and Breeding of Distinctive Cannabis, Ronin Publishing, ISBN 091417178X, 9780914171782), Short (2004, Cultivating Exceptional Cannabis: An Expert Breeder Shares His Secrets, ISBN 1936807122, 9781936807123), Cervantes (2004, Marijuana Horticulture: The Indoor/Outdoor Medical Grower's Bible, Van Patten Publishing, ISBN 187882323X, 9781878823236), Franck et al. (1990, Marijuana Grower's Guide, Red Eye Press, ISBN 0929349016, 9780929349015), Grotenhermen and Russo (2002, Cannabis and Cannabinoids: Pharmacology, Toxicology, and Therapeutic Potential, Psychology Press, ISBN 0789015080, 9780789015082), Rosenthal (2007, The Big Book of Buds: More Marijuana Varieties from the World's Great Seed Breeders, ISBN 1936807068, 9781936807062), Clarke, RC (Cannabis: Evolution and Ethnobotany 2013 (In press)), King, J (Cannabible Vols 1-3, 2001-2006), and four volumes of Rosenthal's Big Book of Buds series (2001, 2004, 2007, and 2011), each of which is herein incorporated by reference in its entirety for all purposes.

Pedigree selection, where both single plant selection and mass selection practices are employed, may be used for the generating varieties as described herein. Pedigree selection, also known as the “Vilmorin system of selection,” is described in Fehr, Walter; Principles of Cultivar Development, Volume I, Macmillan Publishing Co., which is hereby incorporated by reference. Pedigree breeding is used commonly for the improvement of self-pollinating crops or inbred lines of cross-pollinating crops. Two parents which possess favorable, complementary traits are crossed to produce an F1. An F2 population is produced by selfing one or several F1's or by intercrossing two F1's (sib mating). Selection of the best individuals usually begins in the F2 population; then, beginning in the F3, the best individuals in the best families are usually selected. Replicated testing of families, or hybrid combinations involving individuals of these families, often follows in the F4 generation to improve the effectiveness of selection for traits with low heritability. At an advanced stage of inbreeding (e.g., F6 and F7), the best lines or mixtures of phenotypically similar lines are tested for potential release as new cultivars.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection.

Mass and recurrent selections can be used to improve populations of either self- or cross-pollinating crops. A genetically variable population of heterozygous individuals may be identified or created by intercrossing several different parents. The best plants may be selected based on individual superiority, outstanding progeny, or excellent combining ability. Preferably, the selected plants are intercrossed to produce a new population in which further cycles of selection are continued.

Backcross breeding has been used to transfer genes for a simply inherited, highly heritable trait into a desirable homozygous cultivar or line that is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent may be selected and repeatedly crossed (backcrossed) to the recurrent parent. The resulting plant is expected to have the attributes of the recurrent parent (e.g., cultivar) and the desirable trait transferred from the donor parent.

A single-seed descent procedure refers to planting a segregating population, harvesting a sample of one seed per plant, and using the one-seed sample to plant the next generation. When the population has advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

Mutation breeding is another method of introducing new traits into Cannabis varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

The complexity of inheritance also influences the choice of the breeding method. Backcross breeding may be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Additional breeding methods have been known to one of ordinary skill in the art, e.g., methods discussed in Chahal and Gosal (Principles and procedures of plant breeding: biotechnological and conventional approaches, CRC Press, 2002, ISBN 084931321X, 9780849313219), Taji et al. (In vitro plant breeding, Routledge, 2002, ISBN 156022908X, 9781560229087), Richards (Plant breeding systems, Taylor & Francis US, 1997, ISBN 0412574500, 9780412574504), Hayes (Methods of Plant Breeding, Publisher: READ BOOKS, 2007, ISBN1406737062, 9781406737066), each of which is incorporated by reference in its entirety for all purposes. Cannabis genome has been sequenced (Bakel et al., The draft genome and transcriptome of Cannabis sativa, Genome Biology, 12(10):R102, 2011). Molecular markers for Cannabis plants are described in Datwyler et al. (Genetic variation in hemp and marijuana (Cannabis sativa L.) according to amplified fragment length polymorphisms, J Forensic Sci. 2006 March; 51(2):371-5), Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007), Datwyler et al., (Genetic Variation in Hemp and Marijuana (Cannabis sativa L.) According to Amplified Fragment Length Polymorphisms, J Forensic Sci, March 2006, 51(2):371-375), Gilmore et al. (Isolation of microsatellite markers in Cannabis sativa L. (marijuana), Molecular Ecology Notes, 3(1):105-107, March 2003), Pacifico et al., (Genetics and marker-assisted selection of chemotype in Cannabis sativa L.), Molecular Breeding (2006) 17:257-268), and Mendoza et al., (Genetic individualization of Cannabis sativa by a short tandem repeat multiplex system, Anal Bioanal Chem (2009) 393:719-726), each of which is herein incorporated by reference in its entirety for all purposes.

The production of double haploids can also be used for the development of homozygous varieties in a breeding program. Double haploids are produced by the doubling of a set of chromosomes from a heterozygous plant to produce a completely homozygous individual. For example, see Wan et al., Theor. Appl. Genet., 77:889-892, 1989.

### Marker Assisted Selection Breeding

In an embodiment, marker assisted selection (MAS) is used to produce plants with desired traits. MAS is a powerful shortcut to selecting for desired phenotypes and for introgressing desired traits into cultivars (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

Introgression refers to the transmission of a desired allele of a genetic locus from one genetic background to another, which is significantly assisted through MAS. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like.

The introgression of one or more desired loci from a donor line into another is achieved via repeated backcrossing to a recurrent parent accompanied by selection to retain one or more loci from the donor parent. Markers associated with powdery mildew resistance may be assayed in progeny and those progeny with one or more desired markers are selected for advancement. In another aspect, one or more markers can be assayed in the progeny to select for plants with the genotype of the agronomically elite parent. This invention anticipates that trait introgressed resistance to powdery mildew will require more than one generation, wherein progeny are crossed to the recurrent (agronomically elite) parent or selfed. Selections are made based on the presence of one or more resistance to powdery mildew markers and can also be made based on the recurrent parent genotype, wherein screening is performed on a genetic marker and/or phenotype basis. In another embodiment, markers of this invention can be used in conjunction with other markers, ideally at least one on each chromosome of the Cannabis genome, to track the resistance to powdery mildew phenotypes.

Genetic markers are used to identify plants that contain a desired genotype at one or more loci, and that are expected to transfer the desired genotype, along with a desired phenotype to their progeny. Genetic markers can be used to identify plants containing a desired genotype at one locus, or at several unlinked or linked loci (e.g., a haplotype), and that would be expected to transfer the desired genotype, along with a desired phenotype to their progeny. The present invention provides the means to identify plants that exhibit resistance to powdery mildew by identifying plants having powdery resistance specific markers.

In general, MAS uses polymorphic markers that have been identified as having a significant likelihood of co-segregation with a desired trait. Such markers are presumed to map near a gene or genes that give the plant its desired phenotype, and are considered indicators for the desired trait, and are termed QTL markers. Plants are tested for the presence or absence of a desired allele in the QTL marker.

Genomic selection is another form of marker-assisted selection in which a very large number of genetic markers covering the whole genome are used. With genomic selection, all SNPs are included, each with a different level of effect, in a model to explain the variation of the trait. Genomic selection is based on the analysis of many SNPs, for example tens of thousands or even millions of SNPs. This high number of SNP markers is used as input in a genomic prediction formula that predicts the desired phenotype for MAS.

Identification of plants or germplasm that include a marker locus or marker loci linked to a desired trait or traits provides a basis for performing MAS. Plants that comprise favorable markers or favorable alleles are selected for, while plants that comprise markers or alleles that are negatively correlated with the desired trait can be selected against. Desired markers and/or alleles can be introgressed into plants having a desired (e.g., elite or exotic) genetic background to produce an introgressed plant or germplasm having the desired trait. In some aspects, it is contemplated that a plurality of markers for desired traits are sequentially or simultaneously selected and/or introgressed. The combinations of markers that are selected for in a single plant are not limited, and can include any combination of markers disclosed herein or any marker linked to the markers disclosed herein, or any markers located within the QTL intervals defined herein.

In some embodiments, a first Cannabis plant or germplasm exhibiting a desired trait (the donor) can be crossed with a second Cannabis plant or germplasm (the recipient, e.g., an elite or exotic Cannabis, depending on characteristics that are desired in the progeny) to create an introgressed Cannabis plant or germplasm as part of a breeding program. In some aspects, the recipient plant can also contain one or more loci associated with one or more desired traits, which can be qualitative or quantitative trait loci. In another aspect, the recipient plant can contain a transgene.

MAS, as described herein, using additional markers flanking either side of the DNA locus provide further efficiency because an unlikely double recombination event would be needed to simultaneously break linkage between the locus and both markers. Moreover, using markers tightly flanking a locus, one skilled in the art of MAS can reduce linkage drag by more accurately selecting individuals that have less of the potentially deleterious donor parent DNA. Any marker linked to or among the chromosome intervals described herein can thus find use within the scope of this invention.

Similarly, by identifying plants lacking a desired marker locus, plants having unfavorable resistance to powdery mildew can be identified and eliminated from subsequent crosses. These marker loci can be introgressed into any desired genomic background, germplasm, plant, line, variety, etc., as part of an overall MAS breeding program designed to enhance resistance to powdery mildew. The invention also provides chromosome QTL intervals that can be used in MAS to select plants that demonstrate different resistance to powdery mildew traits. The QTL intervals can also be used to counter-select plants that have less favorable resistance to powdery mildew.

Thus, the invention permits one skilled in the art to detect the presence or absence of resistance to powdery mildew genotypes in the genomes of Cannabis plants as part of a MAS program, as described herein. In one embodiment, a breeder ascertains the genotype at one or more markers for a parent having favorable resistance to powdery mildew which contains a favorable resistance to powdery mildew allele, and the genotype at one or more markers for a parent with unfavorable resistance to powdery mildew, which lacks the favorable resistance to powdery mildew allele. A breeder can then reliably track the inheritance of the resistance to powdery mildew alleles through subsequent populations derived from crosses between the two parents by genotyping offspring with the markers used on the parents and comparing the genotypes at those markers with those of the parents. Depending on how tightly linked the marker alleles are with the trait, progeny that share genotypes with the parent having resistance to powdery mildew alleles can be reliably predicted to express the desirable phenotype and progeny that share genotypes with the parent having unfavorable resistance to powdery mildew alleles can be reliably predicted to express the undesirable phenotype. Thus, the laborious, inefficient, and potentially inaccurate process of manually phenotyping the progeny for resistance to powdery mildew traits is avoided.

Closely linked markers flanking the locus of interest that have alleles in linkage disequilibrium with resistance to powdery mildew alleles at that locus may be effectively used to select for progeny plants with desirable resistance to powdery mildew traits. Thus, the markers described herein, such as those listed in Tables 1 and 2, as well as other markers genetically linked to the same chromosome interval, may be used to select for Cannabis plants with different resistance to powdery mildew traits. Often, a set of these markers will be used, (e.g., 2 or more, 3 or more, 4 or more, 5 or more) in the flanking regions of the locus. Optionally, as described above, a marker flanking or within the actual locus may also be used. The parents and their progeny may be screened for these sets of markers, and the markers that are polymorphic between the two parents used for selection. In an introgression program, this allows for selection of the gene or locus genotype at the more proximal polymorphic markers and selection for the recurrent parent genotype at the more distal polymorphic markers.

In an embodiment, MAS is used to select one or more cannabis plants comprising resistance to powdery mildew, the method comprising: i) obtaining nucleic acids from a sample plant or its germplasm; (ii) detecting one or more markers that indicate resistance to powdery mildew, and (iii) indicating resistance to powdery mildew,

A number of SNPs together within a sequence, or across linked sequences, can be used to describe a haplotype for any particular genotype (Ching et al. (2002), BMC Genet. 3:19 pp Gupta et al. 2001, Rafalski (2002b), Plant Science 162:329-333). Haplotypes may in some circumstances be more informative than single SNPs and can be more descriptive of any particular genotype. Haplotypes of the present invention are described in Table 5, and can be used for marker assisted selection.

The choice of markers actually used to practice the invention is not limited and can be any marker that is genetically linked to the intervals as described herein, which includes markers mapping within the intervals. In certain embodiments, the invention further provides markers closely genetically linked to, or within approximately 0.5 cM of, the markers provided herein and chromosome intervals whose borders fall between or include such markers, and including markers within approximately 0.4 cM, 0.3 cM, 0.2 cM, and about 0.1 cM of the markers provided herein.

In some embodiments the markers and haplotypes described above can be used for marker assisted selection to produce additional progeny plants comprising the indicated resistance to powdery mildew. In some embodiments, backcrossing may be used in conjunction with marker-assisted selection.

### Gene Editing

In some embodiments gene editing is used to develop plants having powdery mildew resistance. In particular, methods for selecting one or more cannabis plants having resistance to powdery mildew, the method comprising: (i) replacing a nucleic acid sequence of a parent plant with a nucleic acid sequence conferring resistance to powdery mildew, (ii) crossing or selfing the parent plant, thereby producing a plurality of progeny seed, and (iii), selecting one or more progeny plants grown from the progeny seed that comprise the nucleic acid sequence conferring resistance to powdery mildew, thereby selecting modified plants having resistance to powdery mildew.

Gene editing is well known in the art, and many methods can be used with the present invention. For example, a skilled artisan will recognize that the ability to engineer a trait relies on the action of the genome editing proteins and various endogenous DNA repair pathways. These pathways may be normally present in a cell or may be induced by the action of the genome editing protein. Using genetic and chemical tools to over-express or suppress one or more genes or elements of these pathways can improve the efficiency and/or outcome of the methods of the invention. For example, it can be useful to over-express certain homologous recombination pathway genes or suppression of non-homologous pathway genes, depending upon the desired modification.

For example, gene function can be modified using antisense modulation using at least one antisense compound, including antisense DNA, antisense RNA, a ribozyme, DNAzyme, a locked nucleic acid (LNA) and an aptamer. In some embodiments the molecules are chemically modified. In other embodiments the antisense molecule is antisense DNA or an antisense DNA analog.

RNA interference (RNAi) is another method known in the art to reduce gene function in plants, which is mediated by RNA-induced silencing complex (RISC), a sequence-specific, multicomponent nuclease that destroys messenger RNAs homologous to the silencing trigger. RISC is known to contain short RNAs (approximately 22 nucleotides) derived from the double-stranded RNA trigger. The short-nucleotide RNA sequences are homologous to the target gene that is being suppressed. Thus, the short-nucleotide sequences appear to serve as guide sequences to instruct a multicomponent nuclease, RISC, to destroy the specific mRNAs. The dsRNA used to initiate RNAi, may be isolated from native source or produced by known means, e.g., transcribed from DNA. Plasmids and vectors for generating RNAi molecules against target sequence are now readily available from commercial sources.

DNAzyme molecules, enzymatic oligonucleotides, and mutagenesis are other commonly known methods for reducing gene function. Any available mutagenesis procedure can be used, including but not limited to, site-directed point mutagenesis, random point mutagenesis, in vitro or in vivo homologous recombination (DNA shuffling), uracil-containing templates, oligonucleotide-directed mutagenesis, phosphorothioate-modified DNA mutagenesis, mutagenesis using gapped duplex DNA, point mismatch repair, repair-deficient host strains, restriction-selection and restriction-purification, deletion mutagenesis, total gene synthesis, double-strand break repair, zinc-finger nucleases (ZFN), transcription activator-like effector nucleases (TALEN), any other mutagenesis procedure known to a person skilled in the art.

A skilled artisan would also appreciate that clustered regularly interspaced short palindromic repeats (CRISPR)/CRISPR associated protein (Cas) system comprises genome engineering tools based on the bacterial CRISPR/Cas prokaryotic adaptive immune system. This RNA-based technology is very specific and allows targeted cleavage of genomic DNA guided by a customizable small noncoding RNA, resulting in gene modifications by both non-homologous end joining (NHEJ) and homology-directed repair (HDR) mechanisms (Belhaj K. et al., 2013. Plant Methods 2013, 9:39). In some embodiments, a CRISPR/Cas system comprises a CRISPR/Cas9 system. CRISPR-based gene editing systems need not be limited to Cas9 systems, as those skilled in the art are aware of other analogous editing enzymes, e.g., MAD7.

Methods for transformation of plant cells required for gene editing are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumeficiens mediated transformation. Transformation means introducing a nucleotide sequence in a plant in a manner to cause stable or transient expression of the sequence.

In plant transformation techniques (e.g., vacuum-infiltration, floral spraying or floral dip procedures) are well known in the art and may be used to introduce expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et at. 1993 C. R. Acad. Sci. 316:1194-1199; Chung et at. 2000 Transgenic Res. 9:471-476; Clough et al. 1998 Plant J. 16:735-743; and Desfeux et at. 2000 Plant Physiol 123:895-904). In these embodiments, seed produced by the plant comprise the expression cassettes encoding the genome editing proteins of the invention. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown, and crossed with the same or different plant varieties using traditional breeding techniques to produce seed, which are then selected under the appropriate conditions.

The expression cassette can be integrated into the genome of the plant cells, in which case subsequent generations will express the genome editing proteins of the invention. Alternatively, the expression cassette is not integrated into the genome of the plant's cell, in which case the genome editing protein is transiently expressed in the transformed cells and is not expressed in subsequent generations.

A genome editing protein itself may be introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell. In these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

The genome editing protein can also be expressed in Agrobacterium as a fusion protein, fused to an appropriate domain of a virulence protein that is translocated into plants (e.g., VirD2, VirE2, VirE2 and VirF). The Vir protein fused with the genome editing protein travels to the plant cell's nucleus, where the genome editing protein would produce the desired double stranded break in the genome of the cell. (see Vergunst et al. 2000 Science 290:979-82).

### Products

In an embodiment a cannabis extract or product is disclosed. The product may be any product known in the cannabis arts, and can include, but is not limited to, a kief, hashish, bubble hash, an edible product, solvent reduced oil, sludge, e-juice, or tincture. As used herein, cannabis sludges are solvent-free cannabis extracts made via multigas extraction including the refrigerant 134A, butane, iso-butane and propane in a ratio that delivers a very complete and balanced extraction of cannabinoids and essential oils. Products can also be prepared by any other method known in the art, including but not limited to lyophilization.

Compositions for pulmonary administration also include, but are not limited to, dry powder compositions consisting of the powder of a cannabis oil described herein, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The cannabis oils can be formulated, for example, us suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, ilipe butter, tamanu butter, carnauba wax, emulsifying wax, soy wax, castor wax, rice bran wax, and candelila wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions set forth herein can be formulated for parenteral administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

The compositions can be prepared according to conventional mixing, granulating, and/or coating methods, and contain from about 0.1 to about 75%, preferably from about 1 to about 50%, of the cannabis oil extract. In general, subjects receiving a cannabis oil composition orally are administered doses ranging from about 1 to about 2000 mg of cannabis oil. A small dose ranging from about 1 to about 20 mg can typically be administered orally when treatment is initiated, and the dose can be increased (e.g., doubled) over a period of days or weeks until the maximum dose is reached.

### Kits for Use in Diagnostic Applications

Kits for use in diagnostic, research, and prognostic applications are also provided by the invention. Such kits may include any or all of the following: assay reagents, buffers, nucleic acids for detecting the target sequences and other hybridization probes and/or primers. The kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), cloud-based media, and the like. Such media may include addresses to internet sites that provide such instructional materials.

### EXAMPLES

Aspects of the present teachings can be further understood in light of the following examples, which should not be construed as limiting the scope of the present teachings in any way.

The practice of the present teachings employ, unless otherwise indicated, conventional methods of protein chemistry, biochemistry, recombinant DNA techniques and pharmacology, within the skill of the art. Such techniques are explained fully in the literature. See, e.g., T. Creighton, Proteins: Structures and Molecular Properties, 1993, W. Freeman and Co.; A. Lehninger, Biochemistry, Worth Publishers, Inc. (current addition); J. Sambrook et al., Molecular Cloning: A Laboratory Manual, 2nd Edition, 1989; Methods In Enzymology, S. Colowick and N. Kaplan, eds., Academic Press, Inc.; Remington's Pharmaceutical Sciences, 18th Edition, 1990, Mack Publishing Company, Easton, Pa.; Carey and Sundberg, Advanced Organic Chemistry, Vols. A and B, 3rd Edition, 1992, Plenum Press.

The practice of the present teachings also employ, unless otherwise indicated, conventional methods of statistical analysis, within the skill of the art. Such techniques are explained fully in the literature. See, e.g., J. Little and D. Rubin, Statistical Analysis with Missing Data, 2nd Edition 2002, John Wiley and Sons, Inc., NJ; M. Pepe, The Statistical Evaluation of Medical Tests for Classification and Prediction (Oxford Statistical Science Series) 2003, Oxford University Press, Oxford, UK; X. Zhoue et al., Statistical Methods in Diagnostic Medicine 2002, John Wiley and Sons, Inc., NJ; T. Hastie et. al, The Elements of Statistical Learning: Data Mining, Inference, and Prediction, Second Edition 2009, Springer, N.Y.; W. Cooley and P. Lohnes, Multivariate procedures for the behavioral science 1962, John Wiley and Sons, Inc. NY; E. Jackson, A User's Guide to Principal Components 2003, John Wiley and Sons, Inc., NY.

### Example 1—Powdery Mildew Screen

### Background

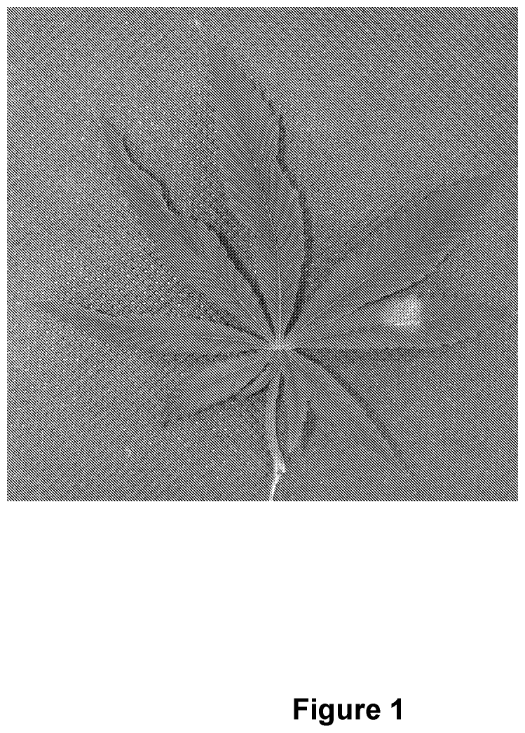

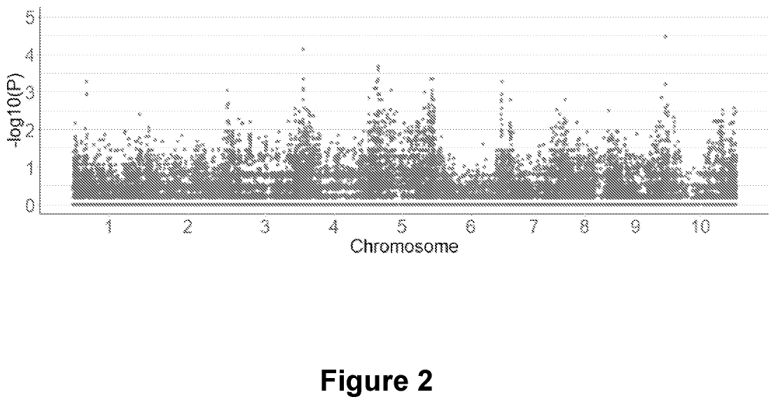

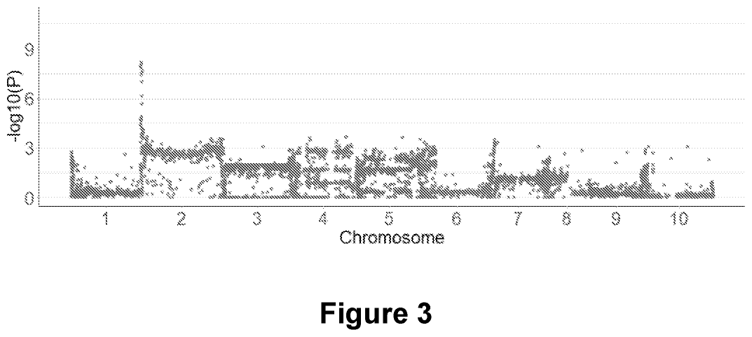

Powdery Mildew, as shown in FIG. 1, can be problematic for cannabis growers. Genetic loci associated with resistance to powdery mildew were mapped in two different experiments. In the first experiment a set of resistant accessions were compared with a set of susceptible accessions and resistance was mapped through bulked segregant analysis (BSA). In the second experiment one of the resistant accessions from the first experiment was crossed with an inferred susceptible accession and powdery mildew resistance was mapped in derived F2 populations through logistic regression.

For the first experiment, 73 greenhouse grown Cannabis sativa accessions were evaluated for powdery mildew (Golovinomyces cichoreaceum “sensu lato”) resistance during the summer/fall of 2019 and winter of 2020 using two different assays: whole plant spray assay (WPSA) and detached leaf conidia transfer assay (DLA) as well as based on observations of powdery mildew infection during various natural outbreaks in the greenhouse. All resistant accessions as well as some of the susceptible accessions were evaluated using both assays.

WPSA involved a spray inoculation of young plants that were three weeks old. The spray inoculum was prepared by mixing eight small leaves with visible powdery mildew growth in 150 ml water. After preparation the inoculum was immediately used to spray the plants which were grown inside a tent with an environment conducive to powdery mildew growth. After 10 days the first powdery mildew symptoms started to appear, at which time each plant was carefully evaluated for any signs of powdery mildew infection. Powdery mildew symptoms, regardless of severity, as well as whether a plant was symptom free, were noted. During the first two weeks after onset of symptoms, screening for powdery mildew was performed twice a week. A final recording of symptoms was made during the third week after onset of the first symptoms. In total 1-3 accessions per seed lot for 23 seed lots were evaluated. Each accession was grown at 1-7 clonal replicates. In total 73 accessions were evaluated.

Plants of the WPSA assay were scored for their levels of susceptibility (S=mildly susceptible, SS=intermediate levels of susceptibility,SSS=highly susceptible) and resistance (R=mildly resistant, RR=intermediate level of resistance, RRR=highly resistant) based on the number of clonal replicates showing signs of powdery mildew lesions as well as the date of onset of infection. Symptoms visible as early as 10-14 days past inoculation are an indication of high susceptibility (SSS). Accessions where none of the clonal replicates showed any sign of powdery mildew lesions were marked as highly resistant (RRR). Accessions where less than half the clonal replicates showed signs of powdery mildew lesions after 15 days were marked as mildly resistant (R). Accessions where first symptoms were visible as early as 10 days, but less than half the number of clonal replicates had symptoms for the first three time points (through day 15 after inoculation) were marked mildly susceptible (S). Plants were scored for the intermediate categories (SS and RR) if they had symptoms between mild and severe manifestations of the disease for both level of susceptibility and resistance, respectively. The seven accessions that showed no powdery symptoms (RRR) in the WPSA were extensively tested by DLA. After DLA only three of these seven accessions remained highly resistant (RRR) and the status of the remaining four was modified to mildly susceptible.

The DLA was performed in the lab and involved transfer of clumps of conidia. Leaves that were used to transfer the conidia to were most recently fully expanded leaves from greenhouse grown plants. Three leaves were placed in a double dish petri plate with the petioles sticking through the top plate into media on the bottom plate. Each petri dish was previously filled with 0.4% water agar with 200 mg/L benzimidazole. Conidia were transferred once to the middle blade of each leaf. The petri dishes with leaves were placed on shelves under 16 hours light. Subsequently, 15 days after inoculation each leaf was evaluated for severity of powdery mildew symptoms and susceptibility/resistance scores similar to the WPSA were recorded. For the DLA, only size of the powdery mildew lesions was used to assign scores to reflect levels of susceptibility or resistance. Accessions were marked as highly susceptible (SSS) after confirmation that leaves that were regularly infected and had dense growth at all or most inoculation sites. SS was used when DLA results showed good inoculation success but lesions were not unusually large or dense. S was used when inoculation success was moderate and lesions were small. R was used when inoculation success was around 30% and lesions were small. RR was used when inoculation success was very rare or when all inoculations had such sparse lesions that they could not be seen by the naked eye. RRR was used for leaves with no sign of infection. As a result of this scoring system, S and R categories are very similar and both indicate similar symptoms. All plants initially scored as R were thus converted to S.

Plants in the greenhouse that showed powdery mildew symptoms due to natural outbreaks (=NAT) were always scored as mildly susceptible (S) because it was not always possible without a controlled experiment to distinguish between various levels of susceptibility.

For the second experiment, three F2 populations were created by selfing three F1 plants which were the result of a cross between a powdery mildew resistant accession identified in the first experiment as RRR, and an inferred powdery mildew susceptible accession, based on its SNP marker genotype as identified based on results from the first experiment. Checks included the progeny of the selfed powdery resistant parent and the selfed progeny of a confirmed powdery mildew susceptible accession which was identified in the first experiment as SSS. The three F2 populations (n=240) and two checks (n=25) were randomized over two tents with an environment conducive to powdery mildew growth; the experiment took place during fall of 2021.

Plants were spray inoculated 21 days after sowing with a solution prepared from powdery mildew infected leaves mixed in water (see for details the WPSA section above). A second spray inoculation was performed 6 days after the first spray inoculation. First powdery mildew symptoms were observed 14 days after the first inoculation. The first tent was evaluated 18 days after the first inoculation (12 days after the second inoculation; 18 days after the first inoculation), followed by the second tent which was evaluated 20 days after the first inoculation (14 days after the second inoculation; 20 days after the first inoculation). During these evaluations each plant was examined for powdery mildew symptoms, plants with powdery mildew symptoms were identified as susceptible and culled, plants without powdery mildew symptoms were returned to the tents. Both tents were evaluated again for powdery mildew symptoms 23 days after the second inoculation (29 days after the first inoculation), that same day plants were inoculated for a third time. Both tents were subsequently evaluated again for powdery mildew 13 and 33 days after the third inoculation (42 and 62 days after the first inoculation, respectively). In total 64 plants had no powdery symptoms and 201 plants had powdery mildew symptoms by the final evaluation.

### SNP Genotyping

For the first experiment, all 73 accessions were genotyped with an Illumina bead array. After initial marker QC, further filtering steps were performed to filter out known low quality SNPs, SNPs with large numbers of missing values (>50%) and SNPs with a minor allele frequency <1% using vcftools (Danecek et al. Bioinformatics 27.15:2156-2158 (2011)). After these filtering steps, 29,142 array SNPs remained for analysis.

For the second experiment all 265 plants were genotyped with an Illumina bead array. Next, SNP data for the F2 populations were QC checked, further filtering steps were performed to filter out known low quality SNPs, SNPs with large numbers of missing values (>10%) and SNPs with a minor allele frequency <1% using vcftools (Danecek et al. Bioinformatics 27.15:2156-2158 (2011)). After these filtering steps 14,538 array SNPs remained. Principal component analysis (PCA) was performed based on these SNP data using plink (Purcell et al. 81.3: 559-575 (2007)). PCA results revealed three clusters where each cluster represented an F2 population. Two F2 populations were more related as they were separated from the third F2 population along the first principal component explaining 76.2% of the variation. Comparison of the F1 parents with the F2 populations confirmed for each F2 population to be the descendants of their F1 parent by evaluating pihat values using plink (Purcell et al. 81.3: 559-575 (2007)). The two more related F2 populations were used for mapping, whereas the third less related F2 population was used together with the checks for validation.

### Mapping

For the first experiment in total 56 accessions were used for bulked segregant analysis (BSA) where 5 were resistant and 51 susceptible. The BSA involved two Fisher Exact tests (using the software R) of a list of 4×4 tables, one row per SNP, comparing four categories for both tests: 1. Resistant and homozygous reference allele, susceptible and homozygous reference allele, resistant and heterozygous or homozygous alternative allele, susceptible and heterozygous or homozygous alternative allele. 2. Resistant and homozygous alternative allele, susceptible and homozygous alternative allele, resistant and heterozygous or homozygous reference allele, susceptible and heterozygous or homozygous reference allele.

For the second experiment logistic regression was performed for the two closer related F2 populations combined (180 accessions; 11,620 SNPs after marker QC and filtering steps) using the statistical package R.

### Identification of SNP Markers and Candidate Genes

For the first experiment, BSA results were subsequently filtered for a p-value smaller than 0.0015. This set was further reduced by filtering for SNP markers that are homozygous compared to the reference genome for at least one of the resistant accessions, resulting in 25 SNP markers (Table 1).

TABLE 1
Significant markers identified after BSA (n = 56 accessions).

| | | | Genotype | | | |
|---|---|---|---|---|---|---|
| | | | associated | | | Abacus |
| | | | with powdery | | | reference |
| Corresponding | SNP marker | | mildew | Ref | Alt | genome |
| sequence ID | name | p-value | resistance | call | call | position |
| SEQ ID NO: 1 | 157_2302749 | 5.24E−04 | B | A | C | 15,287,266 |
| SEQ ID NO: 2 | 131417_9003 | 1.10E−03 | B | T | A | 15,368,894 |
| SEQ ID NO: 3 | 139802_34420 | 8.94E−04 | A, X | G | A | 95,466,762 |
| SEQ ID NO: 4 | 142603_9373468 | 5.00E−04 | B | G | A | 3,436,566 |
| SEQ ID NO: 5 | 142603_9327121 | 9.00E−04 | B | C | G | 3,502,761 |
| SEQ ID NO: 6 | 119221_7094 | 7.24E−05 | B | G | A | 3,911,993 |
| SEQ ID NO: 7 | un167323_54_55 | 8.00E−04 | B | A | C | 3,946,006 |
| SEQ ID NO: 8 | 142603_8902197 | 5.00E−04 | B | A | G | 3,995,523 |
| SEQ ID NO: 9 | 142603_8835066 | 5.00E−04 | B | C | A | 4,065,757 |
| SEQ ID NO: 10 | 142254_6817791 | 8.00E−04 | B | C | G | 6,578,414 |
| SEQ ID NO: 11 | 142254_5775012 | 8.00E−04 | B | A | G | 7,810,719 |
| SEQ ID NO: 12 | 142254_5428869 | 7.86E−04 | B | T | C | 8,224,119 |
| SEQ ID NO: 13 | 142254_4381771 | 5.24E−04 | B | A | C | 9,503,718 |
| SEQ ID NO: 14 | Cannabis.v1_scf1106- | 5.00E−04 | B | T | C | 10,940,045 |
| | 67588_101 | | | | | |
| SEQ ID NO: 15 | 122973_2375 | 2.58E−04 | B | A | G | 10,995,445 |
| SEQ ID NO: 16 | 102270_114 | 2.00E−04 | B | A | G | 11,233,933 |
| SEQ ID NO: 17 | 142254_2801948 | 1.10E−03 | B | T | C | 11,252,809 |
| SEQ ID NO: 18 | 142606_140210 | 9.00E−04 | B | T | C | 26,170,920 |
| SEQ ID NO: 19 | 141748_981138 | 4.54E−04 | B | G | A | 72,427,511 |
| SEQ ID NO: 20 | 422_3770626 | 8.94E−04 | B | C | T | 73,623,726 |
| SEQ ID NO: 21 | 422_3274289 | 4.54E−04 | B | T | A | 74,208,470 |
| SEQ ID NO: 22 | 164_687988 | 5.00E−04 | B | T | C | 77,146,321 |
| SEQ ID NO: 23 | 164_381925 | 1.10E−03 | A | T | A | 77,468,821 |
| SEQ ID NO: 24 | Cannabis.v1_scf4545- | 3.30E−05 | B | A | G | 980,137 |
| | 23925_100 | | | | | |
| SEQ ID NO: 25 | 142708_44686 | 6.00E−04 | B | G | T | 984,754 |

| | | | | Position left | Position right |
|---|---|---|---|---|---|
| | | | | flanking | flanking |
| Corresponding | | Left flanking | Right flanking | marker | marker |
| sequence ID | chr | marker haplotype | marker haplotype | haplotype (bp) | haplotype (bp) |
| SEQ ID NO: 1 | 1 | 157_2293833 | 157_2306929 | 15,277,564 | 15,291,446 |
| SEQ ID NO: 2 | 1 | 131417_11088 | 130637_273 | 15,366,809 | 15,402,935 |
| SEQ ID NO: 3 | 2 | 123257_4104 | 157602_390 | 95,458,836 | 95,467,337 |
| SEQ ID NO: 4 | 4 | 206886_2701 | 142603_9365870 | 3,388,047 | 3,444,350 |
| SEQ ID NO: 5 | 4 | 142603_9334862 | 142603_9314535 | 3,495,075 | 3,515,283 |
| SEQ ID NO: 6 | 4 | 119221_722 | 142603_8948617 | 3,905,616 | 3,935,114 |
| SEQ ID NO: 7 | 4 | 142603_8946383 | 142603_8929074 | 3,937,348 | 3,954,523 |
| SEQ ID NO: 8 | 4 | 142603_8906815 | 142603_8874744 | 3,990,905 | 4,026,073 |
| SEQ ID NO: 9 | 4 | 142603_8839078 | 142603_8833663 | 4,061,743 | 4,067,160 |
| SEQ ID NO: 10 | 5 | 142254_6832281 | 142254_6778177 | 6,563,925 | 6,626,590 |
| SEQ ID NO: 11 | 5 | 142254_5783829 | 142254_5748651 | 7,801,902 | 7,837,200 |
| SEQ ID NO: 12 | 5 | 136807_14482 | 142254_5421581 | 8,187,388 | 8,231,407 |
| SEQ ID NO: 13 | 5 | 142254_4389264 | Cannabis.v1_scf683- | 9,496,232 | 9,507,458 |
| | | | 108661_101 | | |
| SEQ ID NO: 14 | 5 | 142254_3101568 | 142254_3089784 | 10,930,567 | 10,942,351 |
| SEQ ID NO: 15 | 5 | 142254_3087626 | 73932_1735 | 10,944,510 | 11,018,166 |
| SEQ ID NO: 16 | 5 | 142254_2832338 | 142254_2754905 | 11,217,176 | 11,310,169 |
| SEQ ID NO: 17 | 5 | 142254_2832338 | 142254_2754905 | 11,217,176 | 11,310,169 |
| SEQ ID NO: 18 | 5 | 142606_145592 | 142606_1927369 | 26,164,885 | 26,184,130 |
| SEQ ID NO: 19 | 5 | 141748_941193 | Cannabis.v1_scf959- | 72,387,436 | 72,428,036 |
| | | | 129382_100 | | |
| SEQ ID NO: 20 | 5 | 102960_1457 | 422_3764894 | 73,620,411 | 73,629,456 |
| SEQ ID NO: 21 | 5 | 422_3283032 | 422_3264790 | 74,199,722 | 74,217,967 |
| SEQ ID NO: 22 | 6 | 164_698610 | 164_681734 | 77,135,699 | 77,152,519 |
| SEQ ID NO: 23 | 6 | 164_486126 | 130343_7026 | 77,362,431 | 77,488,204 |
| SEQ ID NO: 24 | X | 142708_37994 | 142708_94895 | 978,062 | 1,034,965 |
| SEQ ID NO: 25 | X | 142708_37994 | 142708_94895 | 978,062 | 1,034,965 |

First column: SNP marker number; Second column: SNP marker name; Third column, logistic regression p-value; Fourth column, genotype associated with resistance to powdery mildew: A = homozygous for reference allele, B = homozygous for alternative allele, X = heterozygous; Fifth column, reference allele call; Sixth column, alternative allele call; Seventh column, Abacus reference genome position. Eighth column, Abacus reference genome chromosome; Ninth column, left flanking SNP of haplotype surrounding SNP marker; Tenth column, right flanking SNP of haplotype surrounding SNP marker; Eleventh column, Abacus reference genome position left flanking SNP of haplotype surrounding SNP marker; Twelfth column, Abacus reference genome position right flanking SNP of haplotype surrounding SNP marker. In this context a haplotype surrounding a significantly associated SNP marker consists of the genomic region flanked by the nearest non-significant SNP on either side of the SNP marker.

The haplotype surrounding each SNP marker was further evaluated to contain candidate genes. A haplotype is the region flanking a SNP marker that is significantly associated with a mapped trait. The boundaries of a haplotype are the nearest flanking SNPs on either side of the SNP marker that no longer have significant association with the mapped trait.

Candidate genes were identified in each haplotype surrounding each SNP marker on the Abacus reference genome (version CsaAba2; Table 1).

The haplotype surrounding SNP marker 157_2302749 is flanked by SNPs 157_2293833 and 157_2306929, located between positions 15,277,564-15,291,446 bp on chromosome 1. This haplotype contains one candidate gene: putative recombination initiation defects 3 (PRD3; AT1G01690; meiotic double strand break formation).

The haplotype surrounding SNP marker 131417_9003 is flanked by SNPs 131417_11088 and 130637_273, located between positions 15,366,809-15,402,935 bp on chromosome 1. This haplotype contains two candidate genes: Cytochrome P450 86A8 (CYP86A8; At2g45970; biosynthesis of lipids for cuticle, affects cuticle permeability, resistance to the fungal pathogen Botrytis cinerea (Bessire, Michael, et al. “A permeable cuticle in Arabidopsis leads to a strong resistance to Botrytis cinerea.” The EMBO journal 26.8 (2007): 2158-2168.)), and a copia-like retrotransposable element.

The haplotype surrounding SNP marker 139802_34420 is flanked by SNPs 123257_4104 and 157602_390, located between positions 95,458,836-95,467,337 bp on chromosome 2. This haplotype contains two candidate genes: Probable indole-3-pyruvate monooxygenase YUCCA11 (YUC11; At1g21430; Involved in auxin biosynthesis) and queuine tRNA-ribosyltransferase catalytic subunit 1 isoform X1 (Cannabis sativa, no Arabidopsis hornolog was found).

The haplotype surrounding SNP marker 142603_9373468 is flanked by SNPs 206886_2701 and 142603_9365870, located between positions 3,388,047-3,444,350 bp on chromosome 4. This haplotype contains six candidate genes: calcium-dependent protein kinase 1/4/10/14/23/30 (CPK1/4/10/14/23/30; calcium-dependent protein kinase; CPK23 is involved in stress response (Shi, Sujuan, et al. “The Arabidopsis calcium-dependent protein kinases (CDPKs) and their roles in plant growth regulation and abiotic stress responses.” International journal of molecular sciences 19.7 (2018): 1900.), embryonic stem cell-specific 5-hydroxymethylcytosine-binding protein (AT2G26470), hypothetical protein AT3G07440, HAUS augmin-like complex subunit (AUG3; AT5G48520; microtubule organization during plant cell division), pathogenic type III effector avirulence factor Avr AvrRpt-cleavage: cleavage site protein (AT5G48500), Putative lipid-transfer protein (DIR1; At5g48485; Putative lipid transfer protein required for systemic acquired resistance (SAR) long distance signaling (Maldonado, Ana M., et al. “A putative lipid transfer protein involved in systemic resistance signalling in Arabidopsis.” Nature 419.6905 (2002): 399-403)).

The haplotype surrounding SNP marker 142603_9327121 is flanked by SNPs 142603_9334862 and 142603_9314535, located between positions 3,495,075-3,515,283 bp on chromosome 4. This haplotype contains two candidate genes: Fimbrin-1/2/3/5/5 (FIM1/2/3/4/5; AT5G48460; actin bundle development), and NTP2 (AT2G40520; unknown function).

The haplotype surrounding SNP marker 119221_7094 is flanked by SNPs 119221_722 and 142603_8948617, located between positions 3,905,616-3,935,114 bp on chromosome 4. This haplotype contains two candidate genes: Chaperone protein dnaJ 11, chloroplastic (J11/DJC23; AT4G36040; unknown function) and DNA-(apurinic or apyrimidinic site) endonuclease 2 (APE2; At4g36050).

The haplotype surrounding SNP marker un167323_54_55 is flanked by SNPs 142603_8946383 and 142603_8929074, located between positions 3,937,348-3,954,523 bp on chromosome 4. This haplotype contains two candidate genes: basic Helix-Loop-Helix 121 (BHLH121; At3g19860; transcription factor) and Calcium-dependent protein kinase 16 (CPK16; AT1G17890; Calcium Dependent Protein Kinase).

The haplotype surrounding SNP marker 142603_8902197 is flanked by SNPs 142603_8906815 and 142603_8874744, located between positions 3,990,905-4,026,073 bp on chromosome 4. This haplotype contains four candidate genes: Protein kinase superfamily protein (AT1G65950), Naringenin,2-oxoglutarate 3-dioxygenase (F3H; At3g51240: flavonoid biosynthesis), Chalcone-flavanone isomerase family protein (AT5G66230), hypothetical protein AT2G13240.

The haplotype surrounding SNP marker 142603_8835066 is flanked by SNPs 142603_8839078 and 142603_8833663, located between positions 4,061,743-4,067,160 bp on chromosome 4. This haplotype contains one candidate gene: RNA demethylase ALKBH9B (ALKBH9B; At2g17970; Dioxygenase that demethylates RNA).

The haplotype surrounding SNP marker 142254_6817791 is flanked by SNPs 142254_6832281 and 142254_6778177, located between positions 6,563,925-6,626,590 bp on chromosome 5. This haplotype contains one candidate gene: G-type lectin S-receptor-like serine/threonine-protein kinase (At5g24080).

The haplotype surrounding SNP marker 142254_5775012 is flanked by SNPs 142254_5783829 and 142254_5748651, located between positions 7,801,902-7,837,200 bp on chromosome 5. This haplotype contains three candidate genes: Tic22-like family protein (AT5G62650) WD repeat-containing protein ATCSA-1(AT1G27840; UV-B tolerance and genome integrity), and DUF21 domain-containing protein (CBSDUF6; At4g33700).

The haplotype surrounding SNP marker 142254_5428869 is flanked by SNPs 136807_14482 and 142254_5421581, located between positions 8,187,388-8,231,407 bp on chromosome 5. This haplotype contains one candidate gene: Polygalacturonase Clade F 3 (PGF3; AT2G23900)

The haplotype surrounding SNP marker 142254_4381771 is flanked by SNPs 142254_4389264 and Cannabis.v1_scf683-108661_101, located between positions 9,496,232-9,507,458 bp on chromosome 5. This haplotype contains two candidate genes: hypothetical protein AT3G07750 and CYP715A1 (AT5G51280).

The haplotype surrounding SNP marker Cannabis.v1_scf1106-67588_101 is flanked by SNPs 142254_3101568 and 142254_3089784, located between positions 10,930,567-10,942,351 bp on chromosome 5. This haplotype contains one candidate gene: snc1-influencing plant E3 ligase reverse genetic screen 4 (SNIPER4; AT3G48880; involved in plant immunity (Huang, Jianhua, Chipan Zhu, and Xin Li. “SCFSNIPER4 controls the turnover of two redundant TRAF proteins in plant immunity.” The Plant Journal 95.3 (2018): 504-515.)).

The haplotype surrounding SNP marker 122973_2375 is flanked by SNPs 142254_3087626 and 73932_1735, located between positions 10,944,510-11,018,166 bp on chromosome 5. This haplotype does not contain genes in the Abacus reference genome. The CBDRx reference genome (https://www.ncbi.nlm.nih.gov/genome/gdv/browser/genome/?id=GCF 900626175.2) contains six candidate genes in this region: LOC115717563 (located between 12,046,739-12,047,898 bp on chromosome 5 of the CBDRx reference genome; uncharacterized protein), LOC115717014 (located between 12,052,783-12,055,690 bp on chromosome 5 of the CBDRx reference genome; cytokinin hydroxylase-like), LOC115715834 (located between 12,078,573-12,081,614 bp on chromosome 5 of the CBDRx reference genome), LOC115717564 (located between 12,084,431-12,085,162 bp on chromosome 5 of the CBDRx reference genome; uncharacterized protein), LOC115717565 (located between 12,096,843-12,097,511 bp on chromosome 5 of the CBDRx reference genome; uncharacterized protein), LOC115716248 (located between 12,102,074-12,118,320 bp on chromosome 5 of the CBDRx reference genome; tripeptidyl-peptidase 2-like).

The haplotype surrounding SNP markers 102270_114 and 142254_2801948 is flanked by SNPs 142254_2832338 and 142254_2754905, located between positions 11,217,176-11,310,169 bp on chromosome 5. This haplotype contains one candidate gene: Phosphate transporter PHO1 homolog 10 (PHO1-H10; At1g69480).

The haplotype surrounding SNP marker 142606_140210 is flanked by SNPs 142606_145592 and 142606_1927369, located between positions 26,164,885-26,184,130 bp on chromosome 5. This haplotype contains one candidate gene: Carotenoid cleavage dioxygenase 7, chloroplastic (CCD7; At2g44990).

The haplotype surrounding SNP marker 141748_981138 is flanked by SNPs 141748_941193 and Cannabis.v1_scf959-129382_100, located between positions 72,387,436-72,428,036 bp on chromosome 5. This haplotype contains one candidate gene: SBP (S-ribonuclease binding protein) family protein (AT1G32740).

The haplotype surrounding SNP marker 422_3770626 is flanked by SNPs 102960_1457 and 422_3764894, located between positions 73,620,411-73,629,456 bp on chromosome 5. This haplotype does not contain genes in the Abacus and CBDRx reference genomes.

The haplotype surrounding SNP marker 422_3274289 is flanked by SNPs 422_3283032 and 422_3264790, located between positions 74,199,722-74,217,967 bp on chromosome 5. This haplotype does not contain genes in the Abacus reference genome. The CBDRx reference genome contains two candidate genes in this region: LOC115718090 (located between positions 83,054,167-83,055,104 on chromosome 5 of the CBDRx reference genome; uncharacterized protein) and LOC115718091 (located between positions 83,067,023-83,068,240 on chromosome 5 of the CBDRx reference genome; uncharacterized protein).

The haplotype surrounding SNP marker 164_687988 is flanked by SNPs 164_698610 and 164_681734, located between positions 77,135,699-77,152,519 bp on chromosome 6. This haplotype contains one candidate gene: hypothetical protein AT4G28025.

The haplotype surrounding SNP marker 164_381925 is flanked by SNPs 164_486126 and 130343_7026, located between positions 77,362,431-77,488,204 bp on chromosome 6. This haplotype contains four candidate genes: tRNA-thr(GGU) m(6)t(6)A37 methyltransferase (AT4G28020), Xyloglucan endotransglucosylase/hydrolase protein 14 (XTH14; AT4G25820), Xyloglucan endotransglucosylase/hydrolase protein 15 (XTH15; AT4G14130), Diacylglycerol kinase 3/4/7 (DGK3/4/7; At4g30340; Phosphorylation of diacylglycerol to generate phosphatidic acid, which is required for response to pathogen attack (Arisz, Steven A., et al. “Rapid phosphatidic acid accumulation in response to low temperature stress in Arabidopsis is generated through diacylglycerol kinase.” Frontiers in plant science 4 (2013): 1.)).

The haplotype surrounding SNP markers Cannabis.v1_scf4545-23925_100 and 142708_44686 is flanked by SNPs 142708_37994 and 142708_94895, located between positions 978,062-1,034,965 bp on chromosome X. This haplotype contains one candidate gene: Acetyl-CoA carboxylase 1 (ACC1; At1g36160; fatty acid biosynthesis, cuticle permeability (Monda, Keina, et al. “Cuticle permeability is an important parameter for the trade-off strategy between drought tolerance and C02 uptake in land plants.” Plant Signaling & Behavior 16.6 (2021): 1908692.)).

In the second experiment, logistic regression based on 180 F2 accessions resulted in 15 SNPs with significant associations with powdery mildew resistance (significance was below the Bonferroni multi-test threshold of 4.31E-06; Table 2). These markers were part of a peak between positions 87,222 and 2,236,802 on chromosome 2 of the Abacus reference genome (version CsaAba2) with most significantly associated SNP markers 141928_959500 at position 396,437 (p=6.58E-09) and 141928_549308 at position 887,683 (p=7.95E-09).

TABLE 2
Significant markers identified after logistic regression based on two
F2 populations which clustered together in PCA (n = 180 accessions).

| | | | Genotype | | | |
|---|---|---|---|---|---|---|
| | | | associated | | | Abacus |
| | | | with powdery | | | reference |
| Corresponding | SNP marker | | mildew | Ref | Alt | genome |
| sequence ID | name | p-value | resistance | call | call | position |
| SEQ ID NO: 26 | Cannabis.v1_scf9338- | 1.25E−08 | B, X | C | A | 119,379 |
| | 6155_113 | | | | | |
| SEQ ID NO: 27 | 141928_1194994 | 1.03E−08 | B, X | T | C | 149,520 |
| SEQ ID NO: 28 | 141928_1163175 | 1.03E−08 | B, X | C | A | 181,346 |
| SEQ ID NO: 29 | 141928_1030083 | 1.10E−08 | B, X | C | G | 325,930 |
| SEQ ID NO: 30 | 141928_959500 | 6.58E−09 | B, X | G | A | 396,437 |
| SEQ ID NO: 31 | 141928_884402 | 1.49E−08 | B, X | C | T | 494,430 |
| SEQ ID NO: 32 | 141928_549308 | 7.95E−09 | B, X | G | A | 887,683 |
| SEQ ID NO: 33 | 141928_329023 | 3.44E−08 | B, X | C | A | 1,114,228 |
| SEQ ID NO: 34 | 141928_179079 | 9.30E−08 | B, X | G | T | 1,262,671 |
| SEQ ID NO: 35 | 141928_100760 | 7.15E−07 | B, X | A | C | 1,368,060 |
| SEQ ID NO: 36 | 141928_44358 | 2.19E−06 | B, X | G | A | 1,430,121 |
| SEQ ID NO: 37 | 141928_39607 | 2.19E−06 | B, X | G | A | 1,434,872 |
| SEQ ID NO: 38 | un18421_41_42 | 2.19E−06 | B, X | C | A | 1,489,951 |
| SEQ ID NO: 39 | 141928_1540244 | 2.48E−08 | B, X | A | C | 1,791,640 |
| SEQ ID NO: 40 | 167_4831479 | 2.37E−08 | B, X | G | A | 2,136,725 |

| | | | | Position left | Position right |
|---|---|---|---|---|---|
| | | | | flanking | flanking |
| Corresponding | | Left flanking | Right flanking | marker | marker |
| sequence ID | chr | marker haplotype | marker haplotype | haplotype (bp) | haplotype (bp) |
| SEQ ID NO: 26 | 2 | 160938_447 | 141928_1183089 | 87,222 | 161,432 |
| SEQ ID NO: 27 | 2 | 160938_447 | 141928_1183089 | 87,222 | 161,432 |
| SEQ ID NO: 28 | 2 | 141928_1171671 | 141928_1151659 | 172,849 | 196,868 |
| SEQ ID NO: 29 | 2 | 141928_1061399 | 141928_967673 | 294,611 | 388,264 |
| SEQ ID NO: 30 | 2 | 141928_967673 | 141928_917652 | 388,264 | 438,385 |
| SEQ ID NO: 31 | 2 | 141928_917652 | 141928_866974 | 438,385 | 511,858 |
| SEQ ID NO: 32 | 2 | 141928_560834 | 141928_547218 | 876,155 | 889,775 |
| SEQ ID NO: 33 | 2 | 141928_354770 | 141928_318263 | 1,088,510 | 1,124,989 |
| SEQ ID NO: 34 | 2 | 105145_638 | 199499_705 | 1,241,220 | 1,266,562 |
| SEQ ID NO: 35 | 2 | 141928_111752 | 141928_95064 | 1,357,068 | 1,373,756 |
| SEQ ID NO: 36 | 2 | 141928_50124 | 141928_30648 | 1,424,355 | 1,443,831 |
| SEQ ID NO: 37 | 2 | 141928_50124 | 141928_30648 | 1,424,355 | 1,443,831 |
| SEQ ID NO: 38 | 2 | 124318_15426 | 110287_33984 | 1,489,815 | 1,506,952 |
| SEQ ID NO: 39 | 2 | 141928_1468941 | 141928_1544892 | 1,687,471 | 1,796,288 |
| SEQ ID NO: 40 | 2 | 141928_1748799 | 167_4737599 | 2,003,496 | 2,236,802 |

First column: SNP marker number; Second column: SNP marker name; Third column, logistic regression p-value; Fourth column, genotype associated with resistance to powdery mildew: A = homozygous for reference allele, B = homozygous for alternative allele, allele, X = heterozygous; Fifth column, reference allele call; Sixth column, alternative allele call; Seventh column, Abacus reference genome position. Eighth column, Abacus reference genome chromosome; Ninth column, left flanking SNP of haplotype surrounding SNP marker; Tenth column, right flanking SNP of haplotype surrounding SNP marker; Eleventh column, Abacus reference genome position left flanking SNP of haplotype surrounding SNP marker; Twelfth column, Abacus reference genome position right flanking SNP of haplotype surrounding SNP marker. In this context a haplotype surrounding a significantly associated SNP marker consists of the genomic region flanked by the nearest non-significant SNP on either side of the SNP marker.

Candidate genes were identified in each haplotype surrounding each SNP marker located on chromosome 2 of the Abacus reference genome (table 2).

The haplotype surrounding SNP markers Cannabis.v1_scf9338-6155_113 and 141928_1194994 is flanked by SNPs 160938_447 and 141928_1183089, located between positions 87,222-161,432 bp on chromosome 2. This haplotype contains 11 candidate genes: Wax ester synthase/diacylglycerol acyltransferase 1 (WSD1; At5g37300; involved in cuticular wax biosynthesis; Li, Fengling, et al. “Identification of the wax ester synthase/acyl-coenzyme A: diacylglycerol acyltransferase WSD1 required for stem wax ester biosynthesis in Arabidopsis.” Plant physiology 148.1 (2008): 97-107), RNA-binding (RRM/RBD/RNP motifs) family protein (AT5G66010), Mannose-P-dolichol utilization defect 1 protein homolog 1 (At5g59470), Transducin/WD40 repeat-like superfamily protein (AT4G14310), Transcription initiation factor TFlID subunit 15 (TAF15; At1g50300; essential for mediating regulation of RNA polymerase transcription), Hexokinase-4 (At3g20040; Fructose and glucose phosphorylating enzyme), Putative pentatricopeptide repeat-containing protein (PCMP-E82; At3g05240; mitochondrial mRNA modification), UDP-Glycosyltransferase superfamily protein (AT5G12890), UDP-glycosyltransferase 92A1 (UGT92A1; AT5G12890), UDP-glycosyltransferase 92A1 (UGT92A1; AT5G12890), Lysine-specific demethylase (JMJ30; At3g20810; Involved in the control of flowering time by demethylating H3K36me2 at the FT locus and repressing its expression), ETHYLENE INSENSITIVE 3-like 3 protein (EIL3: AT1G73730: Probable transcription factor that may be involved in the ethylene response pathway).

The haplotype surrounding SNP marker 141928_1163175 is flanked by SNPs 141928_1171671 and 141928_1151659, located between positions 172,849-196,868 bp on chromosome 2. This haplotype contains 3 genes: 4-coumarate-CoA ligase-like protein (At1g20480; CoA-ligase activity), FT-interacting protein 1 (FTIP1; At5g06850; regulates flowering time under long days), NAD(P)-binding Rossmann-fold superfamily protein (AT3G20790; NADPH regeneration).

The haplotype surrounding SNP marker 141928_1030083 is flanked by SNPs 141928_1061399 and 141928_967673, located between positions 294,611-388,264 bp on chromosome 2. This haplotype contains 4 genes: Lipoxygenase 6, chloroplastic (LOX6; At1g67560; may be involved in pest resistance (Bell, Erin, Robert A. Creelman, and John E. Mullet. “A chloroplast lipoxygenase is required for wound-induced jasmonic acid accumulation in Arabidopsis.” Proceedings of the National Academy of Sciences 92.19 (1995): 8675-8679)), Linoleate 9S-lipoxygenase 1 (LOX1; At1g55020; defense response (Marcos, Ruth, et al. “9-Lipoxygenase-derived oxylipins activate brassinosteroid signaling to promote cell wall-based defense and limit pathogen infection.” Plant physiology 169.3 (2015): 2324-2334.)), Lipoxygenase 2, chloroplastic (LOX2; At3g45140; may be involved in pest resistance (Bell, Erin, Robert A. Creelman, and John E. Mullet. “A chloroplast lipoxygenase is required for wound-induced jasmonic acid accumulation in Arabidopsis.” Proceedings of the National Academy of Sciences 92.19 (1995): 8675-8679)), beta glucosidase 15 (BGLU15; AT2G44450; beta-glucosidase activity).

The haplotype surrounding SNP marker 141928_959500 is flanked by SNPs 141928_967673 and 141928_917652, located between positions 388,264-438,385 bp on chromosome 2. This haplotype contains 8 genes: beta glucosidase 15 (BGLU15; AT2G44450; beta-glucosidase activity), beta glucosidase 17 (BGLU17; AT2G42040; transcription cis-regulatory region binding to AT5G44030 (Taylor-Teeples, Mallory, et al. “An Arabidopsis gene regulatory network for secondary cell wall synthesis.” Nature 517.7536 (2015): 571-575.)), a cellulose synthase involved in secondary cell wall biosynthesis and bacterial and fungal pathogen resistance (Hernendez-Blanco; Camilo, et al. “Impairment of cellulose synthases required for Arabidopsis secondary cell wall formation enhances disease resistance.” The Plant Cell 19.3 (2007): 890-903.)), rho GTPase-activating gacO-like protein (AT3G57930; defense response inferred from genomics data (Depuydt, Thomas, and Klaas Vandepoele. “Multi-omics network-based functional annotation of unknown Arabidopsis genes.” bioRxiv (2021).), hypothetical protein (AT5G59020), CEL-Activated Resistance 1 (CAR1; AT1G50180; immune receptor which recognizes the conserved effectors AvrE and HopAA1 (Laflamme, Bradley, et al. “The pan-genome effector-triggered immunity landscape of a host-pathogen interaction.” Science 367.6479 (2020): 763-768.)), Disease resistance protein (CC-NBS-LRR class) family (AT1G53350), receptor-like protein kinase 1 (RLK1; AT5G60900; defense response to fungus (Brotman, Yariv, et at. “The LysM receptor-like kinase LysM RLK1 is required to activate defense and abiotic-stress responses induced by overexpression of fungal chitinases in Arabidopsis plants.” Molecular plant 5.5 (2012): 1113-1124.)).

The haplotype surrounding SNP marker 141928_884402 is flanked by SNPs 141928_917652 and 141928_866974, located between positions 438,385-511,858 bp on chromosome 2. This haplotype contains 7 genes: receptor-like protein kinase 1 (RLK1; AT5G60900; defense response to fungus (Brotman, Yariv, et al. “The LysM receptor-like kinase LysM RLK1 is required to activate defense and abiotic-stress responses induced by overexpression of fungal chitinases in Arabidopsis plants.” Molecular plant 5.5 (2012): 1113-1124.)), CEL-Activated Resistance 1 (CAR1; AT1G50180; immune receptor which recognizes the conserved effectors AvrE and HopAA1 (Laflamme, Bradley, et al. “The pan-genome effector-triggered immunity landscape of a host-pathogen interaction.” Science 367.6479 (2020): 763-768.)), Disease resistance protein (CC-NBS-LRR class) family (AT1G53350), Disease resistance protein (CC-NBS-LRR class) family (AT1G53350), Cysteine—tRNA ligase 2, cytoplasmic (At5g38830), receptor-like protein kinase 1 (RLK1; AT5G60900; defense response to fungus (Brotman, Yariv, et at. “The LysM receptor-like kinase LysM RLK1 is required to activate defense and abiotic-stress responses induced by overexpression of fungal chitinases in Arabidopsis plants.” Molecular plant 5.5 (2012): 1113-1124.)), Cation efflux family protein (MTP11; AT2G39450; manganese transporter).

The haplotype surrounding SNP marker 141928_549308 is flanked by SNPs 141928_560834 and 141928_547218, located between positions 876,155-889,775 bp on chromosome 2. This haplotype contains 2 genes: Delta(12)-fatty-acid desaturase (FAD2; At3g12120; fatty acid biosynthesis, resistance to fungus resulting from cuticle permeability alterations (Dubey, Olga, et al. “Plant surface metabolites as potent antifungal agents.” Plant Physiology and Biochemistry 150 (2020): 39-48.)), anaphase-promoting complex subunit 8 (APC8; AT3G48150).

The haplotype surrounding SNP marker 141928_329023 is flanked by SNPs 141928_354770 and 141928_318263, located between positions 1,088,510-1,124,989 bp on chromosome 2. This haplotype contains 5 genes: Probable ion channel POLLUX (At5g49960), F-box and associated interaction domains-containing protein (AT3G17570), Protein DMP3 (At4g24310; membrane remodelling), DNA-binding bromodomain-containing protein (AT1G58025), Pseudouridine synthase family protein (AT1G09800).

The haplotype surrounding SNP marker 141928_179079 is flanked by SNPs 105145_638 and 199499_705, located between positions 1,241,220-1,266,562 bp on chromosome 2. This haplotype contains 2 genes: ARM repeat superfamily protein (AT3G03440; defense response to bacterium inferred from genomics data (Depuydt, Thomas, and Klaas Vandepoele. “Multi-omics network-based functional annotation of unknown Arabidopsis genes.” bioRxiv (2021).), resistance protein Ler3 (At5g48620: defense response inferred from genomics data (Depuydt, Thomas, and Klaas Vandepoele. “Multi-omics network-based functional annotation of unknown Arabidopsis genes.” bioRxiv (2021)).

The haplotype surrounding SNP marker 141928_100760 is flanked by SNPs 141928_111752 and 141928_95064, located between positions 1,357,068-1,373,756 bp on chromosome 2. This haplotype contains 4 genes: nuclease (AT5G41980), CCR4-NOT transcription complex subunit (AT5G18420), Protein kinase superfamily protein (AT2G40980), RING/U-box superfamily protein (AT1G47570).

The haplotype surrounding SNP markers 141928_44358 and 141928_39607 is flanked by SNPs 141928_50124 and 141928_30648, located between positions 1,424,355-1,443,831 bp on chromosome 2. This haplotype contains 2 genes: ABC transporter D family member 1 (ABCD1; At4g39850; lipid catabolic process), TOM1-LIKE 5 (TOL5; AT5G63640; ubiquitin binding protein).

The haplotype surrounding SNP marker un18421_41_42 is flanked by SNPs 124318_15426 and 110287_33984, located between positions 1,489,815-1,506,952 bp on chromosome 2. This haplotype contains 2 genes: protection of telomeres 1b (POT1b; AT5G06310; telomere capping), RNA-binding KH domain-containing protein (AT1G09660).

The haplotype surrounding SNP marker 141928_1540244 is flanked by SNPs 141928_1468941 and 141928_1544892, located between positions 1,687,471-1,796,288 bp on chromosome 2. This haplotype contains 12 genes: Cytokinin riboside 5-monophosphate phosphoribohydrolase (LOG5; At4g35190; Cytokinin-activating enzyme), pollen receptor like kinase 3 (PRK3; AT3G42880), glycosyltransferase family protein 2 (AT5G60700), trimethylguanosine synthase (AT2G28310), UDP-xylose transporter 3 (UXT3; At1g06890; nucleotide-sugar transporter), Pleckstrin homology (PH) and lipid-binding START domains-containing protein (AT2G28320; contains region with similarity to EDR2, which is involved in powdery mildew resistance (Vorwerk, Sonja, et al. “EDR2 negatively regulates salicylic acid-based defenses and cell death during powdery mildew infections of Arabidopsis thaliana.” BMC plant biology 7.1 (2007): 1-14.)), spore wall protein 2-like, partial (Cannabis sativa, no significant homology with Arabidop.sis thaliana), Protein-tyrosine sulfotransferase (TPST; At1g08030; innate immune response (Igarashi, Daisuke, Kenichi Tsuda, and Fumiaki Katagiri. “The peptide growth factor, phytosulfokine, attenuates pattern-triggered immunity.” The plant journal 71.2 (2012): 194-204.)), GATA transcription factor 10 (GATA10; AT1G08000; zinc finger transcription factor).

The haplotype surrounding SNP marker 167_4831479 is flanked by SNPs 141928_1748799 and 167_4737599, located between positions 2,003,496-2,236,802 bp on chromosome 2. This haplotype contains 17 genes: transmembrane protein (AT2G28410), zinc finger, C3HC4 type family protein (AT2G28430), zinc finger, C3HC4 type family protein (AT2G28430), zinc finger, C3HC4 type family protein (AT2G28430), zinc finger, C3HC4 type family protein (AT2G28430), zinc finger, C3HC4 type family protein (AT2G28430), zinc finger, C3HC4 type family protein (AT2G28430), zinc finger, C3HC4 type family protein (AT2G28430), C3HC4 type family protein (AT2G28430), Protein disulfide isomerase-like 1-4 (PDIL1-4; At5g60640; protein folding), APAP1 (AT3G39080), 4-hydroxy-3-methylbut-2-en-1-yl diphosphate synthase (ferredoxin), chloroplastic (ISPG; At5g60600; involved in systemic acquired resistance (Gil, Mia José, et al. “The Arabidopsis csb3 mutant reveals a regulatory link between salicylic acid-mediated disease resistance and the methyl-erythritol 4-phosphate pathway.” The Plant Journal 44.1 (2005): 155-166)), DHEP synthase RibB-like alpha/beta domain-containing protein (AT5G60590), RING/U-box superfamily protein (AT5g60580), Zinc finger CCCH domain-containing protein 24 (At2g28450), Probable E3 ubiquitin-protein ligase (LOG2; At3g09770), F-box/kelch-repeat protein (At5g60570), Protein DMP7 (At4g28485; abscission), Serine/threonine-protein kinase GRIK1 (At3g45240; Activates SnRK1.1/KIN10 and SnRK1.2/KIN11 by phosphorylation).

Inheritance of these 15 SNP markers is additive with the homozygous reference allele genotype being most susceptible. Plants with this genotype develop powdery mildew symptoms in general sooner after inoculation. Plants with the heterozygous genotype for these markers are less susceptible, these plants develop powdery mildew symptoms later after inoculation. This pattern that was observed for the main SNP marker 141928_959500 was also observed for the other 14 significant SNP markers (Table 2) in both the two F2 populations used for mapping and the F2 population used for validation (Table 3).

TABLE 3
Percentage of accessions identified with powdery mildew at a given
time point (in days after first inoculation) for the F2 mapping and
validation population, respectively, with homozygous reference and
heterozygous genotype for SNP marker 141928_959500. First column:
number of days after first inoculation by which first powdery
mildew showed up. Second column: percentage of accessions showing
powdery mildew symptoms in the F2 mapping population with
homozygous reference genotype; Third column: percentage of
accessions showing powdery mildew symptoms in the F2 mapping
population with heterozygous genotype; Fourth column: percentage
of accessions showing powdery mildew symptoms in the F2 validation
population with homozygous reference genotype; Fifth column:
percentage of accessions showing powdery mildew symptoms in the
F2 validation population with heterozygous genotype.

| Days after first | Mapping - | Mapping - | Validation - | Validation - |
|---|---|---|---|---|
| inoculation | A | X | A | X |
| 20 | 71% | 29% | 44% | 38% |
| 29 | 73% | 20% | 27% | 53% |
| 42 | 78% | 17% | 44% | 44% |
| 62 | 38% | 56% | 0% | 100% |

### Validation of Markers

Validation of the SNP markers discovered in the first experiment was performed using 17 accessions which were screened for powdery mildew similar to the mapping set and which were genotyped for the same array markers. These accessions had different levels of susceptibility. The validation set confirmed beneficial genotypes for the 17 SNP markers as none of the susceptible accessions contained the genotype observed for the 25 SNP markers in the five resistant accessions.

Next, validation of these SNP markers was performed in the set of 240 F2 accessions assuming that the genotype observed for these 25 SNP markers in the resistant parent of these F2 populations is required for resistance to powdery mildew. Since none of the 240 F2 accessions contained this 25 SNP marker beneficial genotype this data set was not suitable for validation of the combination of these 25 SNP markers.

Validation of the SNP markers discovered in the second experiment was performed with the third F2 population as well as selfed progeny of the resistant accession from the first experiment which was used as parent of the F2s in the second experiment as well as the selfed progeny of a susceptible accession used in the first experiment.

SNP marker 141928_959500 segregated for all three genotypes in the third F2 population used for validation (n=60). Of the 16 homozygous reference allele accessions 15 (94%) developed powdery mildew symptoms, whereas of the 29 heterozygous accessions 21 (72%) developed powdery mildew symptoms and 7 (50%) of the 14 homozygous alternate allele accessions developed powdery mildew symptoms. These results show that the highest incidence of powdery mildew is observed for the homozygous reference allele genotype, with reduced incidence observed for the heterozygous genotype and lowest incidence observed for the homozygous alternate allele genotype. A similar pattern with 5-7 accessions with powdery mildew symptoms having homozygous alternate allele genotype was observed for 10 additional SNP markers (Cannabis.v1_scf9338-6155_113, 141928_884402, 141928_549308, 141928_329023, 141928_179079, 141928_100760, 141928_44358, 141928_39607, un18421_41_42, and 141928_1540244). Four SNP markers (141928_1194994, 141928_1163175, 141928_1030083, and 167_4831479) did not segregate and were fixed for the homozygous alternate allele genotype in all accessions from the third F2 population with powdery mildew symptoms.

None of the 20 accessions derived from the selfed susceptible accession were homozygous alternate allele or heterozygous for SNP marker 141928_959500. Of those 20 accessions, 19 developed powdery mildew symptoms (95% of the plants predicted to be susceptible based on the marker genotype developed powdery mildew). The same pattern was observed for five other SNP markers out of the set of 15 SNP markers that were significant in the second experiment (141928_549308, 141928_100760, 141928_44358, 141928_39607, and un18421_41_42). Five other SNP markers from this set had a similar pattern but one of the accessions displaying powdery mildew symptoms was heterozygous (141928_1194994, 141928_1163175, 141928_329023, 141928_179079, and 167_4831479). The remaining 4 SNP markers in this set were either fixed for the homozygous alternate genotype (141928_1030083) or were homozygous alternate allele in 3-4 of the accessions with powdery mildew symptoms (Cannabis.v1_scf9338-6155_113, 141928_884402, and 141928_1540244).

Of the five accessions derived from a self of the resistant F2 parent four developed powdery mildew. All four were either homozygous reference allele or heterozygous for 141928_959500 (100% of the homozygous reference allele and heterozygous plants developed powdery mildew). The same pattern was observed for four additional SNP markers from the set of 15 significant SNP markers identified in the second experiment (Cannabis.v1_scf9338-6155_113, 141928_884402, 141928_549308, and 141928_329023). The single accession that developed no powdery mildew was homozygous alternate allele for all 15 SNP markers. Four of the SNP markers were homozygous alternate allele in three of the accessions with powdery mildew symptoms (141928_1194994, 141928_1163175, 141928_1030083, and 167_4831479), whereas six SNP markers were homozygous alternate allele in one accession with powdery mildew symptoms (141928_179079, 141928_100760, 141928_44358, 141928_39607, un18421_41_42, and 141928_1540244).

In the set of 56 accessions used in the first experiment only the resistant F2 mapping population parent as well as one accession, which was identified as a naturally occurring susceptible plant, were heterozygous for 141928_959500. Since the susceptible accession with the heterozygous genotype was identified as a naturally occurring powdery mildew infection there is no information about the time of onset of powdery mildew. It is possible that the onset was relatively late, since later onset of powdery mildew tends to be associated with the heterozygous genotype for this marker (Table 3). Of the 51 susceptible accessions 50 (98%) had the homozygous reference allele genotype for SNP marker 141928_959500. Since the resistant accession which was heterozygous for this SNP marker was determined by DLA, powdery mildew incidence was only recorded 15 days after inoculation, therefore the late onset phenotype was not recorded. All of the other 14 SNP markers identified in the second experiment were heterozygous for the resistant F2 parent. The 51 powdery mildew susceptible accessions were mostly (65-94%) homozygous reference allele for these 14 SNP markers.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the invention as defined in the appended claims.

1. A method for selecting one or more plants having resistance to powdery mildew, the method comprising i) obtaining nucleic acids from a sample plant or its germplasm; (ii) detecting one or more markers that indicate resistance to powdery mildew, and (iii) indicating resistance to powdery mildew.

2. The method of claim 1 further comprising selecting the one or more plants indicating resistance to powdery mildew.

3. The method of claim 1 wherein the one or more markers comprises a polymorphism relative to a reference genome at nucleotide position:
(a) 15,287,266 on chromosome 1;
(b) 15,368,894 on chromosome 1;
(c) 95,466,762 on chromosome 2;
(d) 119,379 on chromosome 2
(e) 149,520 on chromosome 2
(f) 181,346 on chromosome 2;
(g) 325,930 on chromosome 2;
(h) 396,437 on chromosome 2;
(i) 494,430 on chromosome 2;
(j) 887,683 on chromosome 2;
(k) 1,114,228 on chromosome 2;
(l) 1,262,671 on chromosome 2;
(m) 1,368,060 on chromosome 2;
(n) 1,430,121 on chromosome 2;
(o) 1,434,872 on chromosome 2;
(p) 1,489,951 on chromosome 2;
(q) 1,791,640 on chromosome 2;
(r) 2,136,725 on chromosome 2;
(s) 3,436,566 on chromosome 4;
(t) 3,502,761 on chromosome 4;
(u) 3,911,993 on chromosome 4;
(v) 3,946,006 on chromosome 4;
(w) 3,995,523 on chromosome 4;
(x) 4,065,757 on chromosome 4;
(y) 6,578,414 on chromosome 5;
(z) 7,810,719 on chromosome 5;
(aa) 8,224,119 on chromosome 5;
(bb) 9,503,718 on chromosome 5;
(cc) 10,940,045 on chromosome 5;
(dd) 10,995,445 on chromosome 5;
(ee) 11,233,933 on chromosome 5;
(ff) 11,252,809 on chromosome 5;
(gg) 26,170,920 on chromosome 5;
(hh) 72,427,511 on chromosome 5;
(ii) 73,623,726 on chromosome 5;
(jj) 74,208,470 on chromosome 5;
(kk) 77,146,321 on chromosome 6;
(ll) 77,468,821 on chromosome 6;
(mm) 980,137 on chromosome X; or
(nn) 984,754 on chromosome X,
wherein the reference genome is the Abacus cannabis reference genome (version CsaAba2).

4. The method of claim 3 wherein the nucleotide position comprises:
(a) on chromosome 1:
(1) a C/C genotype at position 15,287,266; or
(2) an A/A genotype genotype at position 15,368,894,
(b) on chromosome 2:
(1) a G/G or G/A genotype at position 95,466,762;
(2) an A/A or C/A genotype at position 119,379;
(3) a C/C or T/C genotype at position 149,520;
(4) an A/A or C/A genotype at position 181,346;
(5) a G/G or G/C genotype at position 325,930;
(6) an A/A or G/A genotype at position 396,437;
(7) a T/T or T/C genotype at position 494,430;
(8) an A/A or G/A genotype at position 887,683;
(9) an A/A or C/A genotype at position 1,114,228;
(10) a T/T or G/T genotype at position 1,262,671;
(11) a C/C or C/A genotype at position 1,368,060;
(12) an A/A or G/A genotype at position 1,430,121;
(13) an A/A or G/A genotype at position 1,434,872;
(14) an A/A or C/A genotype at position 1,489,951;
(15) a C/C or C/A genotype at position 1,791,640; or
(16) an A/A or G/A genotype at position 2,136,725;
(c) on chromosome 4:
(1) an A/A genotype at position 3,436,566;
(2) a G/G genotype at position 3,502,761;
(3) an A/A genotype at position 3,911,993;
(4) a C/C genotype at position 3,946,006;
(5) a G/G genotype at position 3,995,523; or
(6) an A/A genotype at position 4,065,757;
(d) on chromosome 5:
(1) a G/G genotype at position 6,578,414;
(2) a G/G genotype at position 7,810,719;
(3) a C/C genotype at position 8,224,119;
(4) a C/C genotype at position 9,503,718;
(5) a C/C genotype at position 10,940,045;
(6) a G/G genotype at position 10,995,445;
(7) a G/G genotype at position 11,233,933;
(8) a C/C genotype at position 11,252,809;
(9) a C/C genotype at position 26,170,920];
(10) an A/A genotype at position 72,427,511;
(11) a T/T genotype at position 73,623,726; or
(12) an A/A genotype at position 74,208,470;
(e) on chromosome 6:
(1) a C/C genotype at position 77,146,321; or
(2) a T/T genotype at position 77,468,821,
(f) on chromosome X:
(1) a G/G genotype at position 980,137; or
(2) a T/T genotype at position 984,754,
wherein the reference genome is the Abacus cannabis reference genome (version CsaAba2).

5. The method of claim 1 wherein the one or more markers comprises a polymorphism at position 26 of any one or more of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:25; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; or SEQ ID NO:40.

6. The method of claim 5 wherein the nucleotide position comprises:
(1) a C/C genotype at position 26 of SEQ ID NO:1;
(2) an A/A genotype at position 26 of SEQ ID NO:2;
(3) a G/G or G/A genotype at position 26 of SEQ ID NO:3;
(4) an A/A genotype at position 26 of SEQ ID NO:4;
(5) a G/G genotype at position 26 of SEQ ID NO:5;
(6) an A/A genotype at position 26 of SEQ ID NO:6;
(7) a C/C genotype at position 26 of SEQ ID NO:7;
(8) a G/G genotype at position 26 of SEQ ID NO:8;
(9) an A/A genotype at position 26 of SEQ ID NO:9;
(10) a G/G genotype at position 26 of SEQ ID NO:10;
(11) a G/G genotype at position 26 of SEQ ID NO:11;
(12) a C/C genotype at position 26 of SEQ ID NO:12;
(13) a C/C genotype at position 26 of SEQ ID NO:13;
(14) a C/C genotype at position 26 of SEQ ID NO:14;
(15) a G/G genotype at position 26 of SEQ ID NO:15;
(16) a G/G genotype at position 26 of SEQ ID NO:16;
(17) a C/C genotype at position 26 of SEQ ID NO:17;
(18) a C/C genotype at position 26 of SEQ ID NO:18;
(19) an A/A genotype at position 26 of SEQ ID NO:19;
(20) a T/T genotype at position 26 of SEQ ID NO:20;
(21) an A/A genotype at position 26 of SEQ ID NO:21;
(22) a C/C genotype at position 26 of SEQ ID NO:22;
(23) a T/T genotype at position 26 of SEQ ID NO:23;
(24) a G/G genotype at position 26 of SEQ ID NO:24;
(25) a T/T genotype at position 26 of SEQ ID NO:25;
(26) an A/A or C/A genotype at position 26 of SEQ ID NO:26;
(27) a C/C or T/C genotype at position 26 of SEQ ID NO:27;
(28) a A/A or C/A genotype at position 26 of SEQ ID NO:28;
(29) a G/G or G/C genotype at position 26 of SEQ ID NO:29;
(30) an A/A or G/A genotype at position 26 of SEQ ID NO:30;
(31) a T/T or T/C genotype at position 26 of SEQ ID NO:31;
(32) an A/A or G/A genotype at position 26 of SEQ ID NO:32;
(33) an A/A or C/A genotype at position 26 of SEQ ID NO:33;
(34) a T/T or G/T genotype at position 26 of SEQ ID NO:34;
(35) a C/C or C/A genotype at position 26 of SEQ ID NO:35;
(36) an A/A or G/A genotype at position 26 of SEQ ID NO:36;
(37) an A/A or G/A genotype at position 26 of SEQ ID NO:37;
(38) an A/A or C/A genotype at position 26 of SEQ ID NO:38;
(39) a C/C or C/A genotype at position 26 of SEQ ID NO:39;
(40) an A/A or G/A genotype at position 26 of SEQ ID NO:40;
wherein the reference genome is the Abacus cannabis reference genome (version CsaAba2).

7. The method of claim 1 wherein the one or more markers comprises a polymorphism relative to a reference genome within any one or more haplotypes wherein the haplotypes comprise the region:
(a) on chromosome 1:
(1) between positions 15,277,564 and 15,291,446; or
(2) between positions 15,366,809 and 15,402,935;
(b) on chromosome 2:
(1) between positions 95,458,836 and 95,467,337;
(2) between positions 87,222 and 161,432;
(3) between positions 172,849 and 196,868;
(4) between positions 294,611 and 388,264;
(5) between positions 388,264 and 438,385;
(6) between positions 438,385 and 511,858;
(7) between positions 876,155 and 889,775;
(8) between positions 1,088,510 and 1,124,989;
(9) between positions 1,241,220 1,266,562;
(10) between positions 1,357,068 and 1,373,756;
(11) between positions 1,424,355 and 1,443,831;
(12) between positions 1,489,815 and 1,506,952;
(13) between positions 1,687,471 and 1,796,288;
(14) between positions 2,003,496 and 2,236,802; or
(c) on chromosome 4:
(1) between positions 3,388,047 and 3,444,350;
(2) between positions 3,495,075 and 3,515,283;
(3) between positions 3,905,616 and 3,935,114;
(4) between positions 3,937,348 and 3,954,523;
(5) between positions 3,990,905 and 4,026,073; or
(6) between positions 4,061,743 and 4,067,160;
(d) on chromosome 5:
(1) between positions 6,563,925 and 6,626,590;
(2) between positions 7,801,902 and 7,837,200;
(3) between positions 8,187,388 and 8,231,407;
(4) between positions 9,496,232 and 9,507,458;
(5) between positions 10,930,567 and 10,942,351;
(6) between positions 10,944,510 and 11,018,166;
(7) between positions 11,217,176 and 11,310,169;
(8) between positions 26,164,885 and 26,184,130;
(9) between positions 72,387,436 and 72,428,036;
(10) between positions 73,620,411 and 73,629,456;
(11) between positions 74,199,722 and 74,217,967; or
(e) on chromosome 6:
(1) between positions 77,135,699 and 77,152,519; or
(2) between positions 77,362,431 and 77,488,204;
(f) on chromosome X:
(1) between positions 978,062 and 1,034,965,
wherein the reference genome is the Abacus cannabis reference genome (version CsaAba2).

8. The method of claim 1 wherein the selecting comprises marker assisted selection.

9. The method of claim 1 wherein the detecting comprises an oligonucleotide probe.

10. The method of claim 1 further comprising crossing the one or more plants comprising the indicated resistance to powdery mildew to produce one or more F1 or additional progeny plants, wherein at least one of the F1 or additional progeny plants comprises the indicated resistance to powdery mildew.

11. The method of claim 10 wherein the crossing comprises selfing, sibling crossing, or backcrossing.

12. The method of claim 10 wherein the at least one additional progeny plant comprising the indicated resistance to powdery mildew comprises an F2-F7 progeny plant.

13-14. (canceled)

15. The method of claim 1 wherein the plant comprises a Cannabis plant.

16. A method for selecting one or more plants comprising resistance to powdery mildew, the method comprising replacing a nucleic acid sequence of a parent plant with a nucleic acid sequence conferring resistance to powdery mildew.

17. A plant selected by the method of claim 1.

18. A seed of the plant of claim 17.

19. A tissue culture of cells or a protoplast produced from the plant of claim 17.

20. A plant generated from the tissue culture of claim 19.

21. (canceled)

22. A method of generating a processed cannabis product comprising the use of the plant of claim 17.

23. A cannabis product produced from the plant of claim 17.

24. (canceled)
